(19) United States
(12) Patent Application Publication
(10) Pub. No.: US 2011/0223583 A1
(43) Pub. Date: Sep. 15, 2011

(54) DEVICES AND METHODS FOR PROVIDING CONCENTRATED BIOMOLECULE CONDENSATES TO BIOSENSING DEVICES

(75) Inventors: Neil Gordon, Hampstead (CA); Garry Palmateer, London (CA); George Gelb, London (CA)

(73) Assignee: EARLY WARNING INC., Montreal, QC (CA)

(21) Appl. No.: 13/129,672

(22) PCT Filed: Nov. 24, 2009
(86) PCT No.: PCT/CA2009/001709

§ 371 Date: May 17, 2011

Related U.S. Application Data
(60) Provisional application No. 61/117,337, filed on Nov. 24, 2008.

(51) Int. Cl. C12Q 3/00; C12M 1/34; C12M 1/12; C12M 1/36; C12M 1/42; C12Q 1/70; A61L 2/18

(52) U.S. Cl. 435/3; 435/287.1; 435/283.1; 435/286.5; 435/306.1; 435/289.1; 435/5; 422/28

(57) ABSTRACT

Condensing devices and methods for providing a concentrated biomolecule condensate to one or more biosensing devices are provided. The concentrated biomolecule condensate is obtained from a fluid sample which potentially contains traces of one or more target biomolecules. The fluid sample is first separated into a filtered liquid and a retentate biomolecule condensate. A novel filtering module is provided for this purpose. The target biomolecules in the retentate biomolecule condensate are further separated from unwanted materials using magnetic beads coated with antibodies of the target biomolecules. The beaded biomolecule condensate obtained thereby is processed to extract constituents of the target biomolecules, thereby obtaining the concentrated biomolecule condensate, which is distributed to a biosensing device.

### FIELD OF THE INVENTION

The present invention relates to the field of biosensing, and more particularly concerns biomolecule condensing devices and a method for providing a concentrated biomolecule condensate to any one of a plurality of biosensing devices.

### BACKGROUND

Biological hazards are caused by minute life forms called microorganisms and include certain types of infective bacteria, viruses, protozoa, and substances derived from microorganisms, that invade and grow within other living organisms and cause disease. As the consumption of even a small amount of these pathogens can sicken or kill a living organism, biohazards are taking an enormous toll on humans, animals, the food chain, and the environment. The most effective way to prevent the spread of biohazards is to frequently test for the presence of pathogens in people, animals, insects, surfaces, water, air and the food chain, and then rapidly contain biohazards before transmission occurs. As there is no universal indicator for biohazards, each specific type of pathogenic bacteria, virus, protozoa and other species needs to be tested separately to determine its presence. This has created a demand for specific biotesting.

Detecting and identifying biological materials typically requires a capital-intensive laboratory, specialized equipment, costly materials, labor-intensive processing and highly-trained personnel. Biotesting can take several days or even weeks, as many steps are required, including the collection and transportation of samples to the biotesting laboratory. Because of the limited sensitivity of biotesting techniques, samples need to be processed before testing to increase the number of target biomolecules through time-intensive incubation and amplification techniques such as polymerase chain reaction (PCR). Any time delay is a problem, since pathogens and infectious diseases can spread before the test results are known. Furthermore, the high cost per test limits the number of tests that can be undertaken by government agencies, commercial organizations, and consumers due to budget constraints.

In addition to biotesting laboratories, there is a rapidly growing market focused on the identification of biological materials using biosensors, which are measuring devices that convert a biological interaction into a measurable electrical signal. Biosensors can operate independently of laboratories and be used in portable devices and wireless sensor networks. The lower infrastructure cost, reduced consumption of materials, and ease of use of biosensors can greatly reduce the cost per test when compared to laboratory testing, and will likely be in great demand in the future.

An example of an electrochemical biosensor is described in the Assignee's U.S. patent application Ser. No. 12/216,914 filed on Jul. 11, 2008, the contents of which are incorporated herein by reference. This biosensing device includes at least one working electrode having a systematic array of nano-electrode wires projecting vertically from an electrode pad. The nano-electrode wires all have a same shape and size, and are distributed non-randomly over the electrode pad. Biosensor probes are attached to the nano-electrode wires, each including a bioreceptor selected to bind with a complementary target biomolecule to create a binding event, and an electrochemical transducer transducing this binding event into an electrical signal conducted by the corresponding nano-electrode wire.

The strength of the signal obtained through a biosensing device depends on the concentration of the target biomolecules in the sample provided for biosensing. As even a small number of pathogens can pose a health risk, it is important to ensure that a sufficient quantity of the target biomolecules is included in the sample to which the biosensing device is exposed. This is not always the case for small amounts of fluid extracted directly from a potentially affected larger sample.

There is therefore a need for technology enabling the rapid preparation of target biomolecules for biosensor use that avoids time-intensive incubation and amplification techniques.

### SUMMARY OF THE INVENTION

In accordance with a first aspect of the invention, there is provided a biomolecule condensing device for providing a concentrated biomolecule condensate to at least one biosensing device. The concentrated biomolecule condensate is obtained from a fluid sample potentially containing traces of at least one target biomolecule.

The biomolecule condensing device first includes a filtration module. The filtration module has at least one ultrafiltration assembly for separating the fluid sample into a filtered liquid and a retentate biomolecule condensate containing at least one of the target biomolecule, if present in the fluid sample.

A magnetic bead separation module is further provided for separating the retentate biomolecule condensate into a beaded biomolecule condensate, containing the target biomolecules, and waste materials. The magnetic bead separation module includes magnetic beads coated with antibodies of the at least one target biomolecule so that the target biomolecules in the retentate biomolecule condensate become attached to these magnetic beads.

Finally, a microfluidics module for processing the beaded biomolecule condensate to extract constituents of said target biomolecules therefrom is provided, thereby obtaining the concentrated biomolecule condensate. The microfluidics module enables the distribution of the concentrated biomolecule condensate to one of the at least one biosensing device.

In certain embodiments of the invention, microfluidics reagents and other processes can be placed on each module or, preferably, stored in central locations and then dispensed as required. One portion of the microfluidics module can typically be sanitized and reused with fresh reagents, whereas another portion of the microfluidics module can typically be used only once. A distribution capability is also provided to deliver the concentrated biomolecule condensate to an unused portion of the microfluidics module, and where applicable, unused biosensing device.

In accordance with another aspect of the present invention, there is provided a method for sanitizing the biomolecule condensing device described above. This method includes:

a) adding a sanitizing agent to the filtered liquid obtained through the filtering of the filtering module, thereby obtaining a sanitizing solution;

b) circulating the sanitizing solution through at least one of the filtration module, the magnetic bead separation module and the microfluidics module; and

c) leaving the sanitizing solution in the at least one of the filtration module, the magnetic bead separation module and the microfluidics module, for a soaking period.

In accordance with yet another aspect of the invention, there is also provided a microfluidics module packaged as a replacement cartridge that can be replaced when all of the one-time use microfluidics assemblies and biosensing devices have been used. The replaceable cartridge can include the entire microfluidics module or just the one-time use microfluidics and biosensing devices. For example, a set of one-time use components may be provided, including a filter component hosting a filter housing which contains a plurality of filter membranes, each having a corresponding outlet, and a sensor component hosting a plurality of biosensing devices in equal number to the filter membranes.

In accordance with yet another aspect of the invention, there is provided a condensing method for providing a concentrated biomolecule condensate to at least one biosensing device, the concentrated biomolecule condensate being obtained from a fluid sample potentially containing traces of at least one target biomolecule. The method includes:

a) separating the fluid sample into a filtered liquid and a retentate biomolecule condensate containing at least one of the target biomolecule, if present in the fluid sample;

b) attaching the target biomolecules in the retentate biomolecule condensate to magnetic beads coated with antibodies of the at least one target biomolecule, thereby obtaining a beaded biomolecule condensate, and separating the same from waste materials; and

c) processing the biomolecule condensate to extract constituents of the target biomolecules therefrom, thereby obtaining the concentrated biomolecule condensate, and distributing the same to one of the at least one biosensing device.

Advantageously, embodiments of the method according to the above aspect of the invention provide for detection of the target biomolecules without time-sensitive incubation and amplification techniques.

Furthermore, in accordance with another aspect of the invention, there is provided a filtration module for providing a retentate analyte condensate from a fluid sample potentially containing traces of at least one analyte. The filtration module includes at least one ultrafiltration assembly for separating the fluid sample into a filtered liquid and the retentate analyte condensate. Each ultrafiltration assembly includes:
a sample reservoir;
a filter housing containing an ultrafiltration filter for separating the filtered liquid and retentate analyte condensate, the filter housing having an inlet in fluid communication with said sample reservoir, a liquid outlet for outputting the filtered liquid, and a retentate outlet for outputting the retentate analyte condensate;
a concentration loop for circulating the retentate analyte condensate from the retentate outlet of the filter housing back to the sample reservoir and further circulating the retentate analyte condensate through the filter housing for multiple passes, additional portions of the filtered liquid being removed therefrom at each pass; and
an extraction line for extracting the retentate analyte condensate out of the ultrafiltration assembly after said multiple passes.

In one embodiment, the filtration module includes two such ultrafiltration assemblies, a primary assembly and a secondary assembly, connected in a series to provide the retentate biomolecule concentrate extracted from the primary ultrafiltration assembly to the sample reservoir of the secondary ultrafiltration assembly, to define the fluid sample therein. Advantageously, the filtration module may be used both for biosensing applications and chemical sensing applications.

Other features and advantages of the present invention will be better understood upon a reading of the preferred embodiments thereof, with reference to the appended drawings.

### BRIEF DESCRIPTION OF THE DRAWINGS

FIGS. 1A and 1B show a flow chart generally illustrating a condensing method according to an embodiment of the present invention.

FIG. 2 is a schematic representation of an example of a biosensing device to which the concentrated biomolecule condensate may be provided.

FIG. 3A is a schematic representation of the main modules of a biomolecule condensing device according to an embodiment of the present invention. FIG. 3B is a more detailed schematic representation of a biomolecule condensing device according to an embodiment of the present invention.

FIG. 4 is a schematic representation of a filtration module according to one embodiment of the invention.

FIGS. 5A and 5B are respectively a cross-sectional and a perspective view in partial transparency of a hydrodynamic cavitation device for use in an embodiment of the invention.

FIGS. 6A and 6B show two different examples of a magnetic beads module according to embodiments of the invention.

FIGS. 7A and 7B are schematic representations of a microfluidics module according to embodiments of the invention. FIG. 7C is a flow chart of the processes taking place in the microfluidics and detection modules of a device according to the embodiment of FIG. 7B.

FIG. 8A is an exploded view of a microfluidics module according to an embodiment of the invention. FIG. 8B is a flow chart generally illustrating the steps of a method of fabricating a microfluidics module.

FIG. 9 is a schematic representation of a condensing device including components allowing the sanitizing thereof according to an embodiment of the invention.

### DESCRIPTION OF PREFERRED EMBODIMENTS OF THE INVENTION

Embodiments of the present invention will be described herein below in conjunction with the appended drawings, wherein like reference numerals refer to like elements throughout.

The present invention generally provides methods and devices allowing the processing of a fluid sample which potentially contains traces of at least one target biomolecule, in order to obtain a concentrated biomolecule condensate apt to be provided to one or more biosensing devices.

The starting fluid sample may be embodied by any fluid which may contain target biomolecules to be detected, such as water or other liquids, blood or other bodily fluids, liquefied solids or tissues, or liquefied materials from air or gases. Water samples may for example be obtained from a pressurized source connected to a municipal water network or the like, or an unpressurized source such as a lake. The target biomolecules may be any analyte which one may wish to detect and which is apt to bind with a bioreceptor, as described further below. The present invention may be particularly useful in the context of the detection of pathogens or biohazards such as specific strains of bacterium (e.g., E. coli, Salmonella, Vibrio cholerae), viruses (e.g., Hepatitis A, Norovirus), and protozoa (e.g., Cryptosporidium, Giardia). It is of course understood that the list above is given by way of example only, and is in no way limitative to the scope of the present invention.

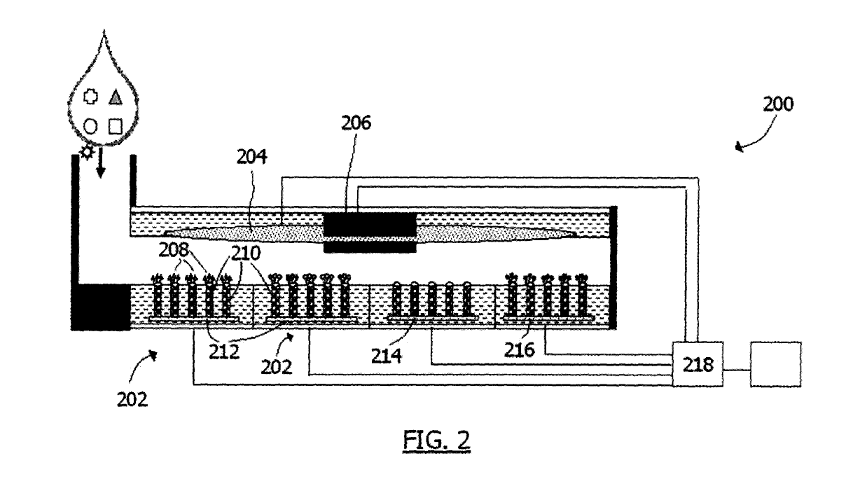

The biosensing devices to which the concentrated biomolecule condensate is provided are preferably embodied by electrochemical sensors, a sensing approach commonly used for the detection of chemicals and in certain cases for the detection of biomolecules. Referring to the enclosed FIG. 2, an example of a biosensing device 200 of this type is shown based on the contents of co-assigned U.S. patent application Ser. No. 12/216,914, filed on Jul. 11, 2008.

The expression “electrochemical sensor” refers to an electrochemical system that determines the presence and concentration of a chemical material or biomaterial through measurements of electrical signal in a solution between a working electrode 202 and counter electrode 204, such as induced by a redox reaction or electrical potential from the release or absorption of ions. The redox reaction refers to the loss of electrons (oxidation) or gain of electrons (reduction) that a material undergoes during electrical stimulation such as applying a potential. Redox reactions take place at the working electrode 202, also referred to as the measuring electrode, and which, for chemical detection, is typically constructed from an inert material such as platinum or carbon. The potential of the working electrode 202 is measured against a reference electrode 206, which is typically a stable, well-behaved electrochemical half-cell such as silver/silver chloride. The electrochemical system can be used to support many different techniques for determining the presence and concentration of the target biomolecules including, but not limited to, various types of voltammetry, amperometry, potentiometry and conductimetry such as AC voltammetry, differential pulse voltammetry, square wave voltammetry, electrochemical impedance spectroscopy, cyclic voltammetry, and fast scan cyclic voltammetry.

The biosensing device 200 of FIG. 2 includes a plurality of working electrodes 202, each having a systematic array of nano-electrode wires 210 projecting vertically from an electrode pad 212. The nano-electrode wires 210 all have a same shape and size and are distributed non-randomly over the electrode pad 212. Biosensor probes 208 are attached to the nano-electrode wires 210. Each biosensor probe 208 includes a bioreceptor selected to bind with a complementary target biomolecule to create a binding event, and an electrochemical transducer transducing this binding event into an electrical signal conducted by the corresponding nano-electrode wire 210. The biosensing device 200 may further include one or more negative control electrode 214 for measuring background noise in the solution, and one or more positive control electrode 216 for measuring a signal from biomolecules known to be present in the solution. Appropriate measurement electronics 218 may also be provided.

In the context of the present invention, one or more biosensing devices may be used, and selected to detect one or more types of target biomolecules. A single biosensing device may be use to detect more than one type of target biomolecule.

In addition to determining whether target biomolecules are present, a given biosensing device may be used to evaluate the concentration of these biomolecules in the solution, as well as the percentage of the cells in the biomolecules that are viable and therefore capable of dividing and increasing in number.

It will be readily understood by one skilled in the art that the condensing devices and methods of embodiments of the present invention may be used in combination with different types of biosensing devices than the one described above. For to example, these can include biosensing devices that measure changes in: temperature (calorimetric biosensors), light output or absorbance (optical biosensors), mass (piezo-electric biosensors), and size, shape and conductivity of a conductive channel in a field effect transistor (field effect biosensors), among others.

### Condensing Method

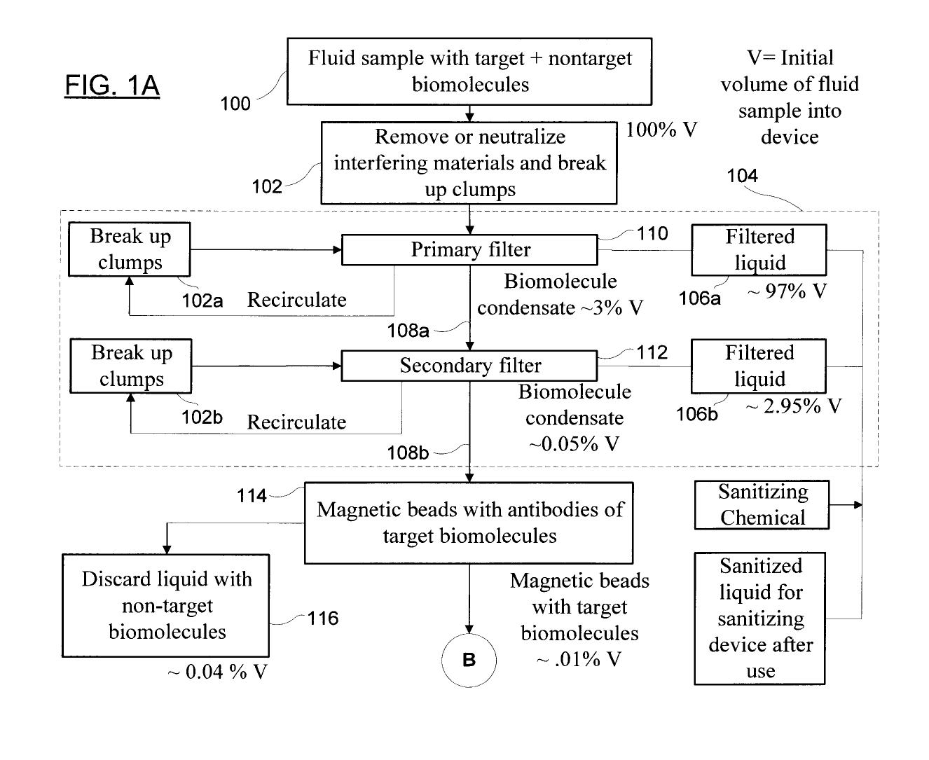

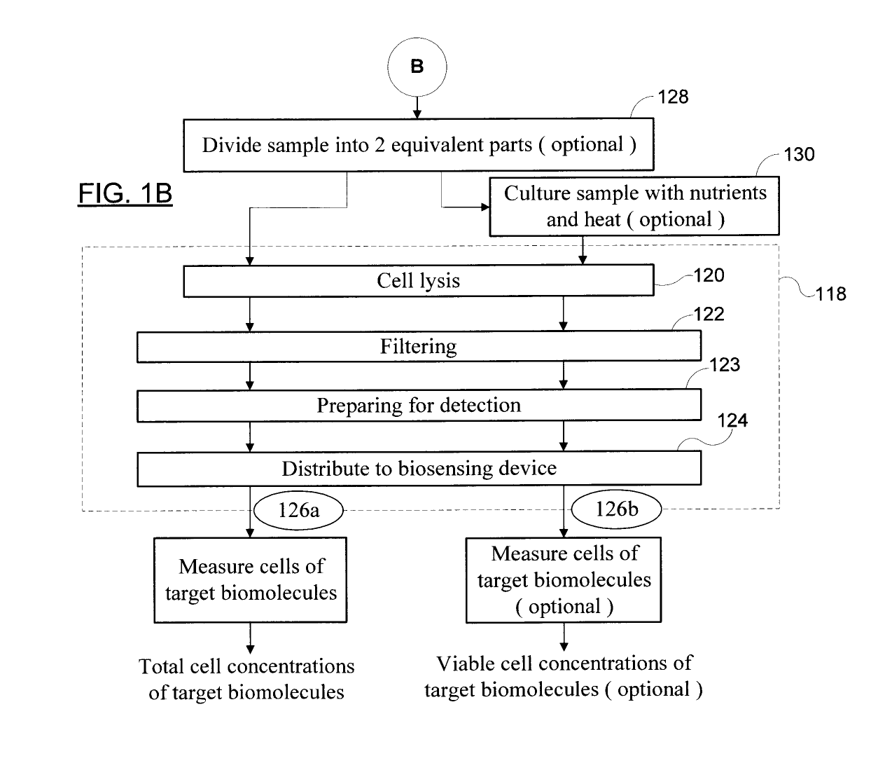

Referring to FIGS. 1A and 1B, a flow chart illustrating the main steps of a condensing method providing a concentrated biomolecule condensate to any one of a plurality of biosensing devices according to an embodiment of the invention is shown. As explained above, the concentrated biomolecule condensate is obtained from a fluid sample 100 potentially containing traces of at least one target biomolecule. The fluid sample may of course include other constituents such as non-target biomolecules, chemicals, and metals. The non-target biomolecules, chemicals and metals may be non-threatening or simply not the object of the sensing being performed. Some of these materials can interfere with the sensing of target biomolecules, and may need to be either neutralized with chemical additives or removed from the solution altogether. Furthermore, the target biomolecules may be attached to or aggregated with other biomolecules or materials in clumps or in biofilms, and the clumps and biofilms may need to be disaggregated to release the target biomolecules prior to sensing to prevent a false negative or understated result.

It will be understood by one skilled in the art that the expression “solution” as used herein is meant to include suspensions or any other form taken by the mixture of the biomolecules and carrying fluid.

The condensing method may include a preliminary pre-treatment step 102 for the liquid solution, such as removing or neutralizing interfering materials and breaking clumps in the fluid sample. Many processes can be employed for this purpose depending on the liquid media; the type, concentration, size and properties of the materials in the liquid; and the environmental conditions such as temperature, pH, etc. In one embodiment, one or more dispensers are provided to add chemicals, such as sodium thiosulfate, to neutralize chlorine in drinking water. An adherent could also be employed to remove interfering materials. In the same embodiment, one or more disaggregation techniques such as surfactants, sonication or preferably hydrodynamic cavitation can also be employed to reduce clumping.

The method next includes separating 104 the fluid sample 100, after the pre-treatment step 102, into a filtered liquid 106 which is substantially free of biomolecules and a retentate biomolecule condensate 108 containing the target biomolecules, if present in the fluid sample. According to one embodiment, this separating involves passing the fluid sample successively though ultrafiltration assemblies, preferably including a primary filter 110 and a secondary filter 112, which may for example both be embodied by tangential flow filters. At each filtering substep, the fluid sample is received in a sample reservoir, circulated through an ultrafiltration filter for separating the filtered liquid and retentate condensate, and recirculated through the corresponding filter for multiple passes, defining filtering loops, additional portions of filtered liquid being extracted from the retentate biomolecule condensate at each pass. The retentate condensate is then extracted out of the corresponding ultrafiltration assembly. In one example, the primary filtering process may set aside 97% of the initial volume of the fluid sample (V) as the filtered liquid and retain 3% V as the retentate biomolecule condensate.

The secondary filtering may further extract another 2.95% V of filtered liquid, retaining about 0.05% of the initial sample volume. Optionally, additional clump-breaking processes and removing or neutralizing interfering materials steps 102 may be performed during the primary filtering, the secondary filtering or both, preferably as part of the corresponding filtering loops.

It will be noted from the above description of the separation in the filtration module 104 of the fluid sample that rather than discarding the biomolecule solution containing bacteria, viruses, protozoa, and other potentially disease causing biomaterials, as is typically done in industrial and medical applications, the biomolecule retentate is returned to the sample reservoir and repeatedly recirculated through the filter, while the filtered liquid, free of biomolecules, is removed. The filtered liquid is preferably passed to a filtered liquid reservoir and, as described below, can be used with a sanitizing agent to sanitize the device between uses.

As the fluid sample containing the biomolecules is repeatedly pumped though the filters, the biomolecules are returned to the input reservoir and the filtered liquid is removed. This greatly changes the concentration of biomolecules since the number of biomolecules stays the same but the volume of solution reduces over time. As a result, the biomolecules become greatly condensed. For example, 1,000 cells contained in 10 liters of solution can be condensed by the primary filter to 1,000 cells in 300 mL of solution, after 9,700 mL of filtered solution (or 97% of the liquid) is removed. As may be expected, some biomolecules may attach to the filter or related systems, and somewhat reduce the yield.

Multiple filtration circuits can be used depending on the volume of the input fluid sample and the volume of the condensate to be recovered after filtration. In one embodiment, 10 liters of potable water is used to condense down to about 1 to 10 mL of condensate for a reduction in volume of 99.9% to 99.99%. In this case, two filtration circuits are provided in a series sequence. A primary filtration circuit condenses the input volume to about 100 to 500 mL of retentate, which is fed into a secondary filtration circuit that further condenses the output to about 1 to 10 mL.

The method next involves a step 114 of mixing the retentate biomolecule condensate 108 resulting from the filtering step 104 with magnetic beads coated with antibodies of at least one target biomolecule and permitting the target biomolecules in the retentate biomolecule condensate to attach to the matching antibodies. One or more solutions can be added to the mix, such as filtered liquid extracted from the filtering step, a buffer solution, or a re-suspension. The mixture is then circulated to a condensation chamber and a beaded biomolecule condensate is obtained when a magnetic field is activated to retain the magnetic beads in the condensation chamber and the waste solution is removed. Once this step is done, the magnetic field is removed with a magnetic insulator, and a rinsing solution and/or compressed gas can bring the magnetic bead retentate to the microfluidics module. In one embodiment, the volume of the magnetic bead retentate is about 0.5-1 mL.

The purpose of the magnetic beads is to separate target biomolecules that will be detected by the biosensing device from non target biomolecules that can interfere with the detection, and need to be removed from the biomolecule retentate and discarded. In the example given above, the separation step condenses a 10 L fluid sample to approximately 5 mL after two filtration circuits. When analyzing potable water, the number of biomolecules per 10 L can be in the thousands or millions of cells. Up to 100% of the cells can be non-pathogenic heterotrophic species that do not need to be detected, and should not be sent to the biosensing device. In this case, the magnetic bead separation is provided to extract the target biomolecules using magnetic beads with antibodies selected to match a suite of target biomolecules commonly identified as waterborne pathogens. These can include E. coli (Indicator), E. coli O157:H7, Campylobacter, Cryptosporidium, Giardia, Enterovirus, etc. The remaining liquid incorporating the non-target biomolecules is preferably discarded 116. Other suites of target biomolecules can be used for different applications such as in testing pathogens for Listeria bacteria in meat or Hepatitis A virus in blood.

The method finally includes a step of processing 118 the beaded biomolecule condensate to extract constituents of the target biomolecules, thereby obtaining a retentate biomolecule condensate which is distributed to an associated biosensing device. Referring to the enclosed FIG. 2, showing an example of a biosensing device 200, in one embodiment, the biosensing devices are used once and then discarded to avoid contamination from the previous sample. In a preferred embodiment, there are multiple biosensing devices available in a cartridge, and the microfluidics module prepares the biomolecule constituents for biodetection at one of the unused biosensing devices.

Preferably, processing step 118 is performed in a microfluidics module. Preferably, the processing first includes lysing cells 120 of the biomolecule condensate to release the biomolecule constituents. In one example, the beaded condensate is pumped to a microfluidics chamber and a cell lysis reagent is added. The lysis reagent and sample are mixed in a mixing chamber and the temperature is maintained at a proper value in a range of 25 to 37° C., to open the cell walls and release the cell constituents. In some lysis methods, the temperature could be as high as 90° C. Optionally, additional processing could be performed to separate the biomolecule constituents from the cell walls. In one embodiment, sonication is used for this purpose; that is, ultrasonic energy is projected through the beaded biomolecule condensate. In another embodiment, the beaded biomolecule condensate is submitted to an alternating low and high temperature cycle, to break open cell walls. This technique may for example be employed for cryptosporidium oocysts that may be more resistant to conventional cell lysis. A binding buffer and washing buffer may be added to improve the extraction and collection of target constituents.

The beaded biomolecule condensate is then filtered 122 to separate the biomolecule constituents from the magnetic beads and waste materials left over from the cell lysis. For this purpose, the mixture is preferably pumped through a membrane and waste material is discarded. An elution buffer is pumped through the membrane and carries off the target constituents.

In one embodiment, for example for 16S ribosomal RNA detection, the method next includes a step of preparing 123 the biomolecule constituents for detection. This may for example involve mixing the biomolecule constituents for DNA digestion with a RW buffer before DNase, DNase and RDD, RPE buffer (trademarks of the Qiagen company) and Ethanol. The temperature may be raised to approximately 70° C. to provide denaturing and unfolding the RNA strands in the biomolecule constituents. The biomolecule constituent may further be mixed with a wash buffer immediately prior to distribution 124 to one of the biosensing devices.

The microfluidics module can further support the biosensing device for temperature control of 25 to 65° C. that may be required for hybridization, and adding other materials such as chemical mediators and positive control target biomolecules. At this stage, the biosensing device is activated to measure the cell concentrations of target biomolecules.

In a preferred embodiment, the beaded biomolecule condensate is divided 128 into two equivalent portions by a metering system in the microfluidics module. One of the portions of the sample is immediately processed as above, and then provided to an available biosensing derive. The other portion of the sample is pumped to a culture chamber and cultured 130 with growth medium, heat and other requirements to encourage any viable cells to reproduce in number. Once a predetermined period of time or condition is attained, the second sample is then sent to an available microfluidics chamber and the above process is repeated so that the second biosensing device can calibrate the difference between the first and second readings, to determine the viability of the target biomaterials in the sample as described in the co-assigned U.S. patent application Ser. No. 12/216,914, filed on Jul. 11, 2008.

### Condensing Device

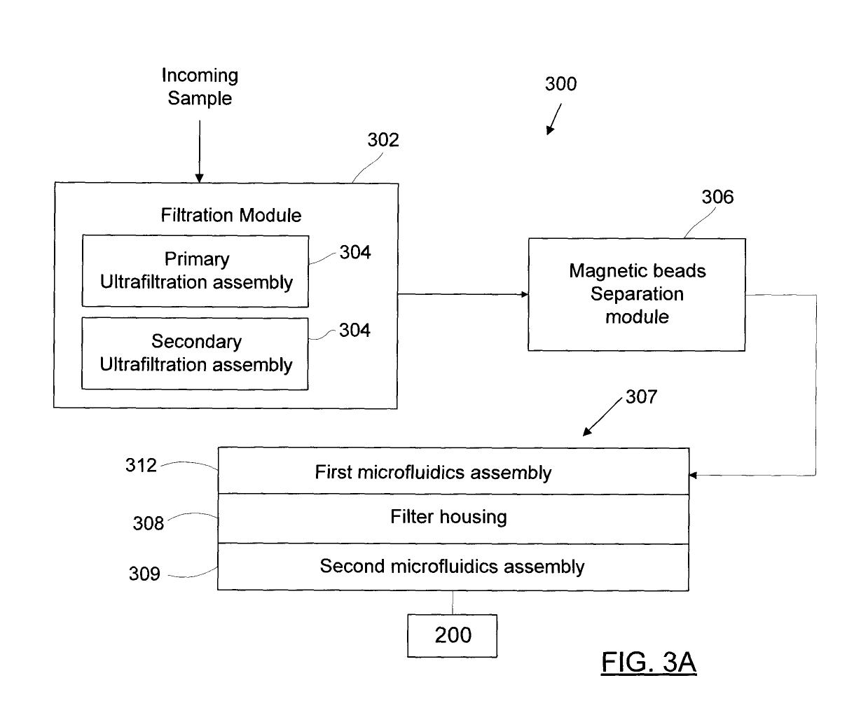

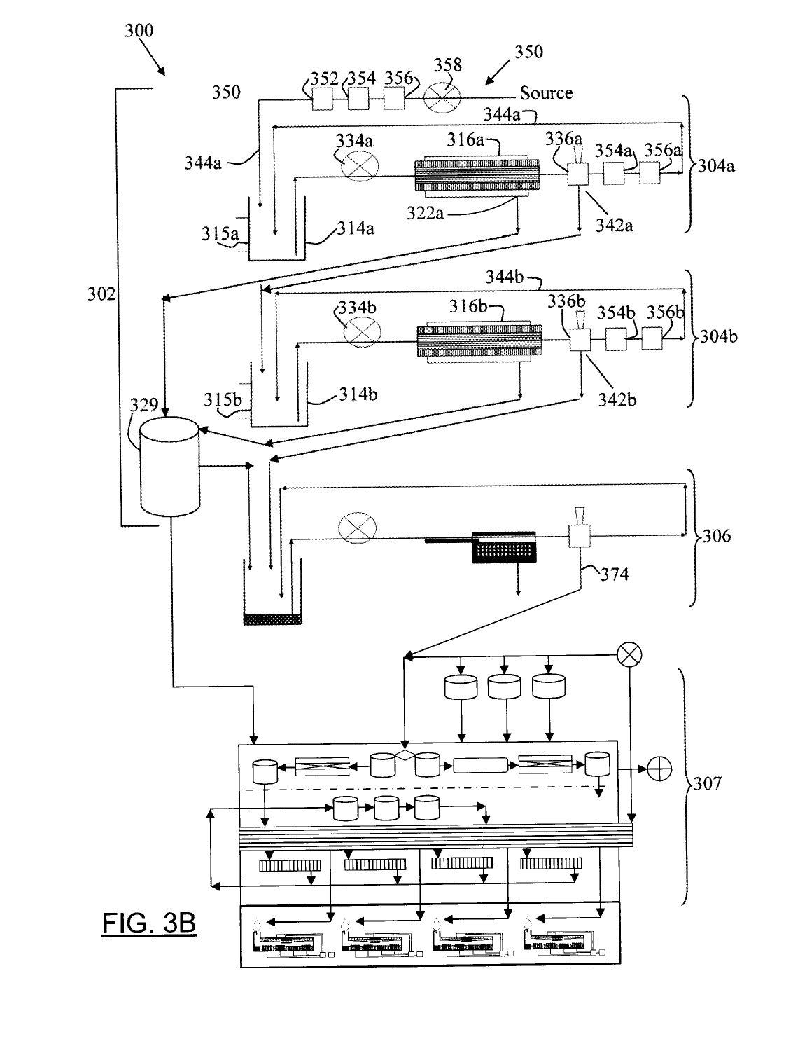

With reference to FIG. 3A, the main modules of a condensing device 300 according to an embodiment of the invention are shown. The biomolecule condensing device 300 first includes a filtration module 302 having one, two or more ultrafiltration assemblies 304 for separating the fluid sample into a filtered liquid and a retentate biomolecule condensate. The retentate biomolecule condensate contains most of the target biomolecules, if present in the fluid sample. A magnetic bead separation module 306 is further provided and includes magnetic beads coated with antibodies of the target biomolecules, so that the target biomolecules in the retentate biomolecule condensate become attached to these magnetic beads. A beaded biomolecule condensate is thereby obtained.

The biomolecule condensing device 300 further includes a microfluidics module 307, which processes the beaded biomolecule condensate to extract constituents of the target biomolecules, thereby obtaining the concentrated biomolecule condensate. Preferably, the microfluidics module 307 includes a first microfluidics assembly 312 which hosts cell lysing means for lysing cells of the biomolecules attached to the magnetic beads of the beaded biomolecule condensate. Various components and processes which may be used for this purpose will be described further below. Cell lysis opens the cell walls and releases the biomolecule constituents of the target biomolecules, i.e. the strands of nucleic acid of the biomolecules. In order to separate these constituents from the magnetic bead, cell walls and other waste material from the cell lysis, the beaded biomolecule condensate from the first microfluidics assembly is then receive in a filter housing 308 which contains one or more filter membranes for retaining waste material from the cell lysis and allowing the biomolecule constituents therethrough. The biomolecule constituents are then received in a second microfluidics assembly 309 which hosts preparation means for the preparation of the biomolecule constituents for detection. This may involve several processes which will also be described further below. The biomolecule constituents are then ready for distribution to an unused biosensor 200, for detection.

In some embodiments, the biomolecule condensing device's pumps, pipes, valves and other components can be configured to support a sanitizing method as described further below, operated either automatically with Programmable Logic Controllers (PLCs) or manually by an operator to direct and control the flow of the liquids, additives and processes.

Each module of condensing device 300 according to embodiments of the invention will now be described in more detail.

### Filtration Module

As mentioned above, the condensing device 300 includes a filtration module 302 which separates the fluid sample into a retentate biomolecule condensate and filtered liquid, the retentate condensate being extracted for further processing and eventual detection of the biomolecules it contains. Although the filtration module 302 is described hereinbelow in the context of biosensing, one skilled in the art will understand that a similar module could be use for the condensation of any soluble analyte, whether biological or chemical. The analyte could for example be embodied by pathogens, drugs, pesticides, industrial chemicals, metals and natural toxic compounds. All the components of the filtration module 312 described below could therefore be adapted for chemical sensing without departing from the scope of this aspect of the present invention.

In the embodiments of FIGS. 3A and 3B, the filtration module 302 includes a primary and a secondary ultrafiltration assembly 304a and 304b. Ultrafiltration is useful to separate viruses, bacteria and protozoa from a solution. When the smallest target biomolecules are viruses at around 50 nanometers in size, filter pores of 50 KiloDaltons are preferably used to capture the viruses along with all larger sized target biomolecules such as bacteria and protozoa. The primary and secondary ultrafiltration assemblies are preferably connected in a series, so as to provide the retentate biomolecule concentrate extracted from the primary ultrafiltration assembly to the secondary ultrafiltration assembly, to define the fluid sample therein. It will be understood by one skilled in the art that a single ultrafiltration assembly could suffice in alternate embodiments of the invention, or that more than two may be required. In the case where two or more ultrafiltration assemblies are provided, they may both or all be of a similar construction, or differ in configuration.

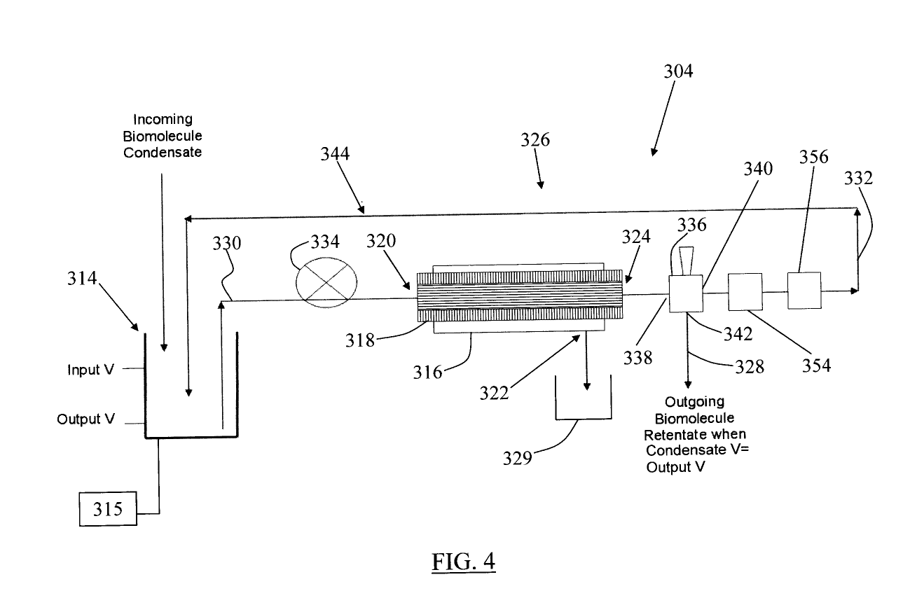

With reference to FIG. 4, there is shown an exemplary representation of an ultrafiltration assembly 304 according to an embodiment of the invention. The ultrafiltration assembly 304 first includes a sample reservoir 314 for receiving the fluid sample. A filter housing 316 containing an ultrafiltration filter 318 for separating the filtered liquid and retentate biomolecule condensate is further provided, the filter housing 316 having an inlet 320 for receiving the fluid sample, one or more liquid outlets 322 for outputting the filtered liquid, and a retentate outlet 324 for outputting the retentate biomolecule condensate. The sample reservoir is in fluid communication with inlet 320 of the filter housing, so that the fluid sample may circulate therebetween.

Throughout the present description, the expression “in fluid communication” is understood to signify that one or more pipe, conduit or any other fluid path connects two components to allow fluid to flow from one to the other, at least in one direction. The communication may be direct or indirect, that is, the fluid may traverse intermediate components during its travel from one component to the other.

In one embodiment, the ultrafiltration filter is a hollow fiber tangential flow filter or membrane filter. Tangential flow filters generally permit a sample solution to flow through a feed channel along the surface of a membrane (tangentially thereto).

Liquid is extracted through the membrane with applied pressure, whereas the particles in the solution remain in the feed channel and are carried along to the retentate outlet. The cross flow prevents build up of molecules at the surface of the membrane, which could cause fouling. This process prevents the rapid decline in flux rate often seen in direct flow filtration, allowing a greater volume to be processed per unit area of membrane surface.

A concentration loop 326 circulates the retentate condensate from the retentate outlet 324 of the filter housing 316 back to the reservoir 314, and further circulates the retentate condensate through the filter housing 316 for multiple passes, additional portions of filtered liquid such as water, liquid food products, beverages, chemicals, urine or blood being removed therefrom at each pass. An extraction line 328 allows the extraction of the retentate biomolecule condensate out of the ultrafiltration assembly 304 after a sufficient number of passes through the ultrafiltration filter 318. This is preferably done when the total volume of solution passing through the sample reservoir 314 is reduced to the desired Output Volume as measured by a sensor 315, flow meter or other suitable devices.

In the illustrated embodiment of FIG. 4, the concentration loop 326 includes an inlet line 330 connecting the sample reservoir 314 and the inlet 320 of the filter housing 316, an outlet line 332 connecting the retentate outlet 324 of the filter housing 316 to the sample reservoir 314, and a pump 334 for cyclically circulating the fluid sample through the concentration loop 326. A valve 336 is provided in the outlet line 332, and has an inlet 338 in fluid communication with the retentate outlet 324 of the filter housing 316, a first outlet 340 for directing the retentate biomolecule condensate to the sample reservoir 314, and a second outlet 342 connected to the extraction line 328.

In this embodiment, once the fluid sample enters the sample reservoir 314, the pump 334 is used to propel the fluid sample though the ultrafiltration filter 318. For example, the fluid sample may be pumped at about 20 to 30 psi into the ultrafiltration filter 318, producing a filtrate of filtered liquid and a retentate containing target and non-target biomolecules. Rather than discarding the retentate, as is typically done in industrial and medical applications, the retentate is returned to the sample reservoir 314 and repeatedly recirculated through the filter 318. Preferably, the filtered liquid is passed to a filtered liquid reservoir 329. As the sample is repeatedly pumped though the ultrafiltration filter 318, the biomolecules are returned to the sample reservoir 314 and the filtered liquid is removed. This greatly changes the concentration of biomolecules since the number of biomolecules stays the same but the volume of solution reduces over time. As a result the biomolecules become greatly condensed. For example, 1,000 cells contained in 10 liters of solution can be condensed to 1,000 cells in 300 mL of solution after 9,700 mL of filtered liquid (or 97% of the liquid) is removed.

Multiple filtration circuits can be used depending on the volume of the fluid in the input sample and the volume of the condensate to be used after filtration. In one embodiment, 10 liters of potable water is used to condense down to about 1 to 10 mL of condensate. In this case, a primary ultrafiltration assembly condenses the input volume to about 100 to 500 mL of retentate, which is fed into a secondary ultrafiltration assembly that further condenses the output to about 1 to 10 mL. Additional rinses with filtered water, buffers and/or air can be employed to release biomolecules attached to the filters or other components of the filtration module.

As mentioned above, FIG. 3B shows a particular embodiment of a filtration module 302 including a primary and a secondary ultrafiltration assembly 304a and 304b. In this embodiment, the fluid sample is obtained directly from a pressurized water supply line. A pre-filtration module 350 is provided to process the fluid sample before it reaches the primary ultrafiltration assembly 304a. The pre-filtration module 350 may include one or more pre-processing filters 352 for filtering the various condensates depending on the composition of the fluid sample. The pre-processing filters 352 may be embodied by a large mesh filter for removing large particles from the fluid sample, such as for example a 40 micron mesh filter. The expression “large” is understood here to refer to particles of a size greater than those containing the biomolecules under study. The pre-processing filter may alternatively or additionally be embodied by a carbon filter for removing chlorine, etc. Various flow controlling devices may be provided as will be readily understood by one skilled in the art, such as valves, pumps, backflow preventers, pressure transducers, regulators, flowmeters, etc. A pump 358 is added in the event that the input solution is not already pressurized as provided to the condensing device 300.

The fluid sample is received in the first sample reservoir 314a. A vent may be to provided to evacuate any air pressure created therein during its fill-up process. Preferably, a sensor 315a is provided in the sample reservoir 314a for sensing a level of the fluid sample therein as the reservoir is being filled. The sensor 315a is operationally connected to both the pump 334a and the valve 336a of the first ultrafiltration assembly 304a. The sensor 315a measures the level of solution against an upper and a lower threshold. When the upper threshold is reached, a valve closes the input loop 344a and the pump 334a is activated to start circulating the fluid sample in the concentration loop of the primary filtration assembly 304a. When a lower threshold level is reached, the valve 336a is set to direct the retentate from the second outlet 342a out to the secondary filtration assembly 304b. The sensor 315a may also be used to control the circulation of the fluid sample in the concentration loop before the sample reservoir 314a is filled with the incoming fluid sample, allowing the ultrafiltration assembly 304 to process an initial volume of fluid greater than the total capacity of the sample reservoir 314a. In this case a flow meter in the input loop 344 can initiate the pump 334a to start while the input sample in still filling sample reservoir 314a and provide the added benefit of reduced processing time.

The fluid sample from the sample reservoir 314a circulates through the primary ultrafiltration assembly 304a for multiple passes through its ultrafiltration filter, as explained above. Preferably, a filtered liquid reservoir 329 is provided and connected to the filtered liquid outlet 322a of the filter housing 316a to receive the filtered liquid that is removed from the liquid solution by the filtration module. Of course, the primary ultrafiltration assembly 304a may include any additional devices typically included in liquid treatment apparatuses, such as valves, pressure gauges and the like.

In the embodiment of FIG. 3B, the secondary ultrafiltration assembly 304b includes similar components as the primary ultrafiltration assembly 304a, such as a sample reservoir 314b with sensor 315b, filter housing 316b, pump 334b (preferably a peristaltic pump in this case), and 3-way valve 336b. The filtered liquid may be extracted to the same filtered liquid reservoir 329 as for the filtered liquid from the primary ultrafiltration assembly, or to a different one. A stirrer may be connected to the sample reservoir 314b, to be activated during the filtration process to improve performance. Of course, the secondary ultrafiltration assembly 304b may also include any additional device typically included in liquid treatment apparatuses, such as valves, pressure gauges and the like.

The filtration module 302 and pre-filtration module 350 may also include one or more processes to break up clumps of biomolecules 354, 354a and 354b. For example, bacteria tend to form aggregates or clumps of materials since cells can naturally attach to other cells as well as to different materials. When a traditional cell culture is done, the output of a Colony Forming Unit (CFU) can actually be 1 cell, or 10 cells, or 100 cells or a clump with an even bigger amount of cells. As a result, traditional cell cultures can understate the true bacteria count because these clumps are not broken up and appear as lower number of CFUs than if the clumps were broken up. In other cases, biomolecules can be trapped in biofilms formed in the incoming fluid sample. Disaggregation techniques such as surfactants, sonication or hydrodynamic cavitation can be added to the prefiltration module and/or each ultrafiltration assembly to reduce clumping.

In one embodiment, hydrodynamic cavitation is used to work like a garden hose to increase the speed of the solution flow and subsequently mechanically erode and decompose the surface of the clumps or biofilms. This will allow the device to break up the clumps when the retentate is recirculated though one or more re-circulation loops and/or pre-filtration module, and ultimately provide a more realistic cell count which will be higher and more accurate.

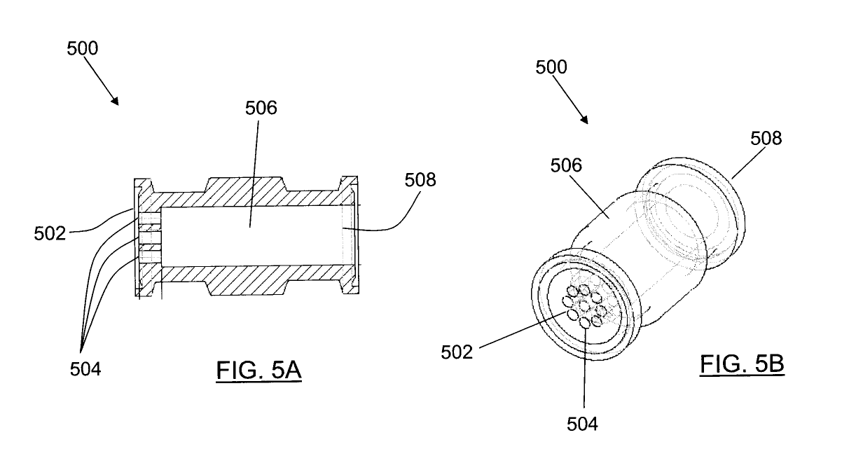

FIGS. 5A and 5B illustrate a hydrodynamic cavitation device 500 which attaches front and back into tubes that permit the flow of liquid potentially containing aggregates of biomolecules. The solution enters the device face 502 containing one or more holes 504 of significantly smaller diameter than the input tube or the device interior 506. The smaller diameter holes have the effect of increasing the liquid velocity as computed by its Reynolds Number or other such calculations. The processed liquid departs the device outlet 508 and is sent to a subsequent process.

Referring back to FIGS. 3B and 4, the filtration module and pre-filtration module can also be modified to provide additives or related processes to remove undesirable materials in the solution that may interfere with the detection of target biomolecules and cause false positive results. These can include additives to neutralize, additives to form a precipitate, or adherents to physically remove undesirable materials. One or more devices 356, 356a and 356b for dispensing chemicals or removing materials depending on the composition of the fluid sample may be provided for this purpose at any point in the filtration module. Such chemicals may for example include additives to disperse biomolecules, such as sodium polyphosphate, and additives to neutralize interfering materials in the fluid sample, such as sodium thiosulfate to neutralize chlorine, etc.

### Magnetic Beads Separation Module

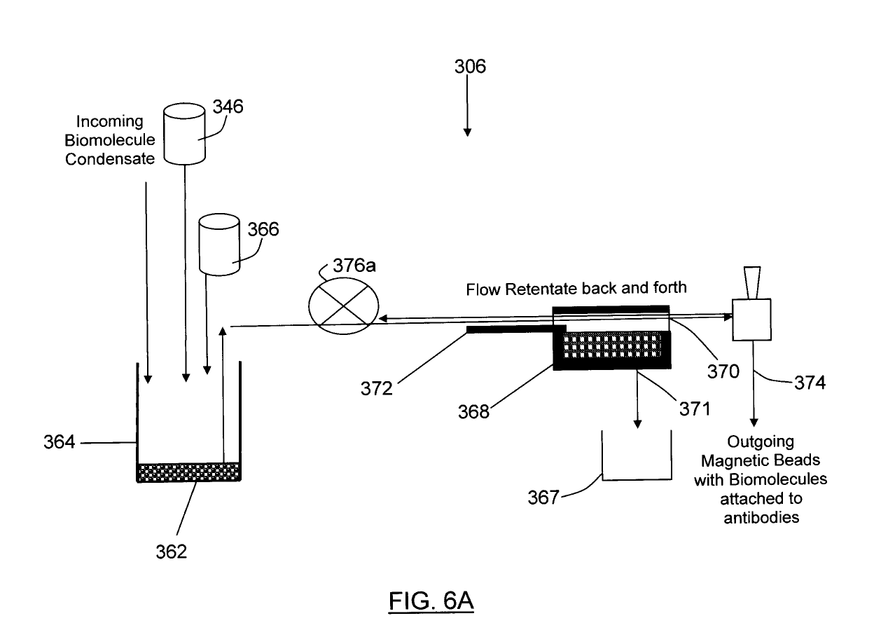

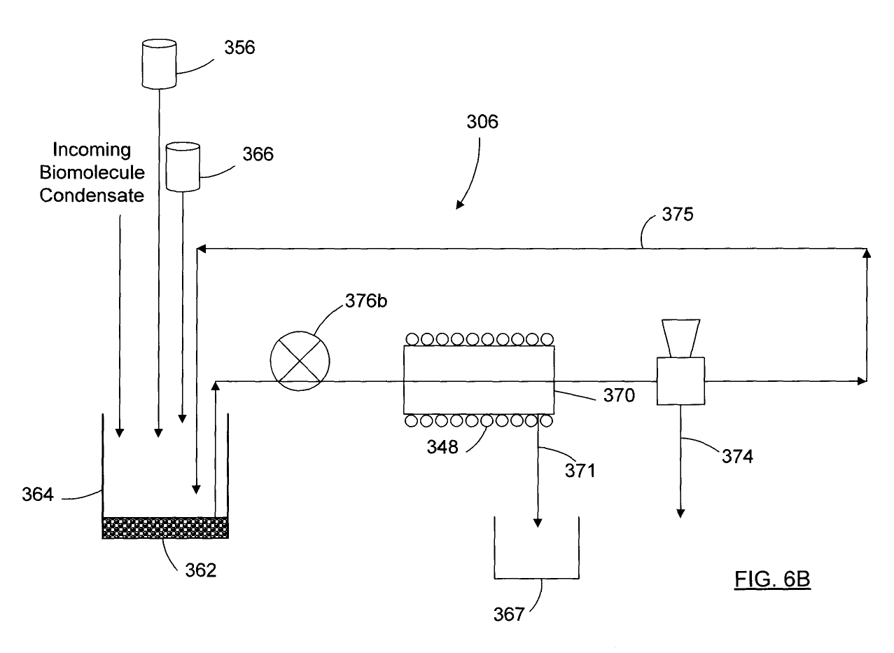

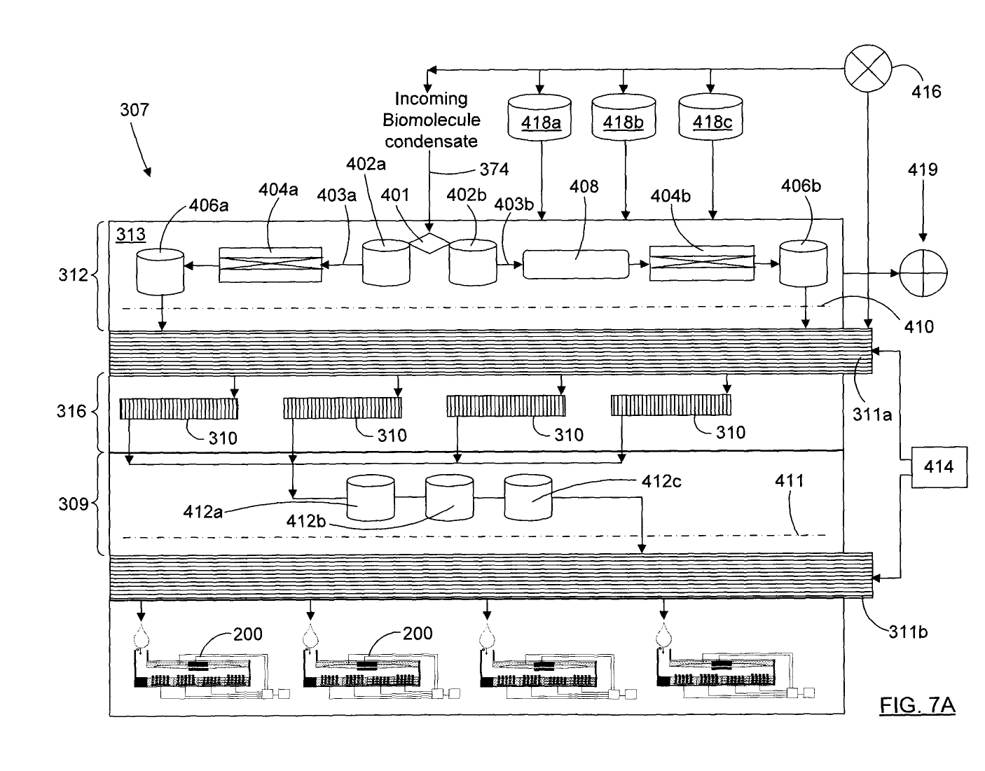

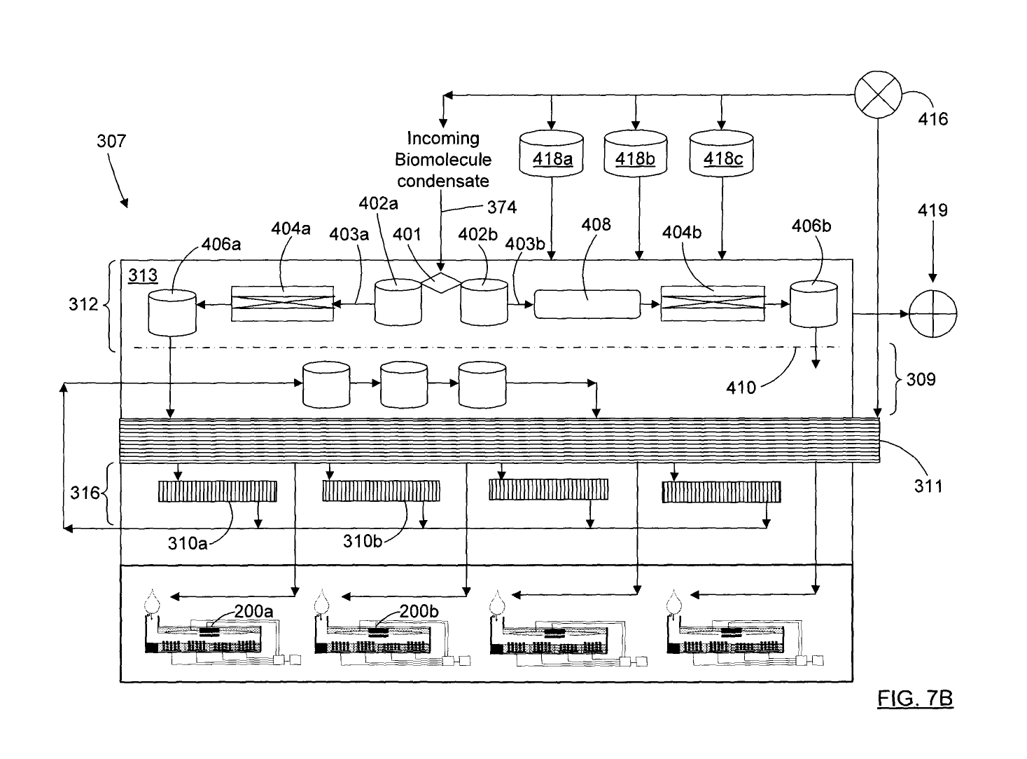

With reference to FIG. 6A, FIG. 6B, and FIG. 3B, there is shown a magnetic beads separation module 306 according to an embodiment of the invention.

The magnetic bead module 306 is preferably provided between the filtration module 302 and the microfluidics module 307. The purpose of the magnetic bead module is to separate target biomolecules that will be detected on a biosensing device 200 from non target biomolecules that can interfere with the detection and need to be discarded. In an embodiment described above, the filtration module condenses a 10 L water sample to approximately 5-7 mL. For example, when sampling potable water, the number of biomolecules per 10 L can be in the thousands or millions of cells. Up to 100% of these biomolecules can be non-pathogenic heterotrophic species that should not be sent to the biosensing device. The magnetic bead module 306 extracts the target biomolecules using magnetic beads 362 with antibodies selected to match a suite of target biomolecules commonly identified as waterborne pathogens. These can include E.coli (Indicator), E.coli O157:H7, Campylobacter, Cryptosporidium, Giardia, Enterovirus, etc. All the magnetic beads 362 mixed with the retentate biomolecule condensate is may be of a same type, or of multiple types depending on the biomolecules to be detected.

In one embodiment, shown in FIG. 6A, the magnetic beads module 306 includes a mixing tank 364 which is connected to the filtration module 302 and receiving therefrom the retentate biomolecule condensate. The appropriate magnetic beads 362 are provided from a beads reservoir 366 into the same mixing tank 364. One or more solution reservoir 346 may be provided in communication with the mixing tank to provide any appropriate solution thereto. For example a buffer solution may be provided in the mixing tank 364 to assist with the capture, and/or a re-suspension solution. A magnetic bead mixture is thereby obtained in the mixing tank 364. The magnetic bead mixture is transferred to a condensation chamber 368, through pumping by a pump 376. Magnetization means are provided to create a magnetic field in the condensation chamber 370, thereby retaining the magnetic beads with the target biomolecules attached thereto in the condensation chamber 370. In the embodiment of FIG. 6A, a permanent magnet 368 is mounted adjacent to the condensation chamber 370 and is used with a constant magnetic field. Unlike variable magnetic fields, the constant field will not induce electrical current and interfere with electrical measurements. The magnet preferably includes a rare earth metal with a strong and highly focused magnetic field that is shielded from the other instruments in the device by the chamber 370. The contact time may for example be from 1-5 minutes, providing an output volume of 0.1-1 mL containing the magnetic beads with any target biomolecules attached to the beads' antibodies. The output solution, defining a beaded biomolecule condensate, is extracted through an extraction line 374 and passed to the microfluidics module 307. In the illustrated embodiment of FIG. 6A, a magnetic insulator 372 is inserted over the magnet after the contact time, thereby disrupting the magnetic field to release the beads with the attached target biomolecules, which can then be pumped to the extraction line 374. In a variant of this embodiment, instead of using a magnetic insulator, the permanent magnet may be moved laterally to drag the magnetic beads, and the attached target biomolecules, towards the extraction line 374. Alternatively, as seen in the embodiment of FIG. 6B, a coil assembly may be mounted around the condensation chamber to induce a magnetic field therein when a current circulates through said coil, interrupting the current releasing the beads. The condensation chamber preferably has a waste outlet 371 for extracting the waste materials resulting from the magnetic bead separation, and the waste outlet is connected to a waste reservoir 367. The waste reservoir 367 may be separate or common to other modules of the condensing device.

The magnetic bead separation is preferably performed at room temperature. It may be necessary to provide refrigeration capabilities for storing the magnetic beads onboard for a finite time between replacement cartridges, such as up to 6 months.

The magnetic bead module may be refined as needed to support other types of solutions such as water drippings from washed fruits; liquefied particles from air; liquefied tissues from plants, animals and humans; blood and other body fluids. Variants to the magnetic beads module described above can include the types of antibodies, the number and size of beads, contact time, magnet type, and mediator depending on the target biomolecules to be detected, the solution type and properties, and the interfering materials in the solution.

The magnetic beads module can be configured to flow the retentate back and forth with pump 376a as in FIG. 6A, or to continuously recirculate the solution with pump 376b as in FIG. 6B. A recirculation assembly 375 may be provided for this purpose, and may have any appropriate structure, such as for example the one described in relation to the concentration loop of the filtration module.

### Microfluidics Module

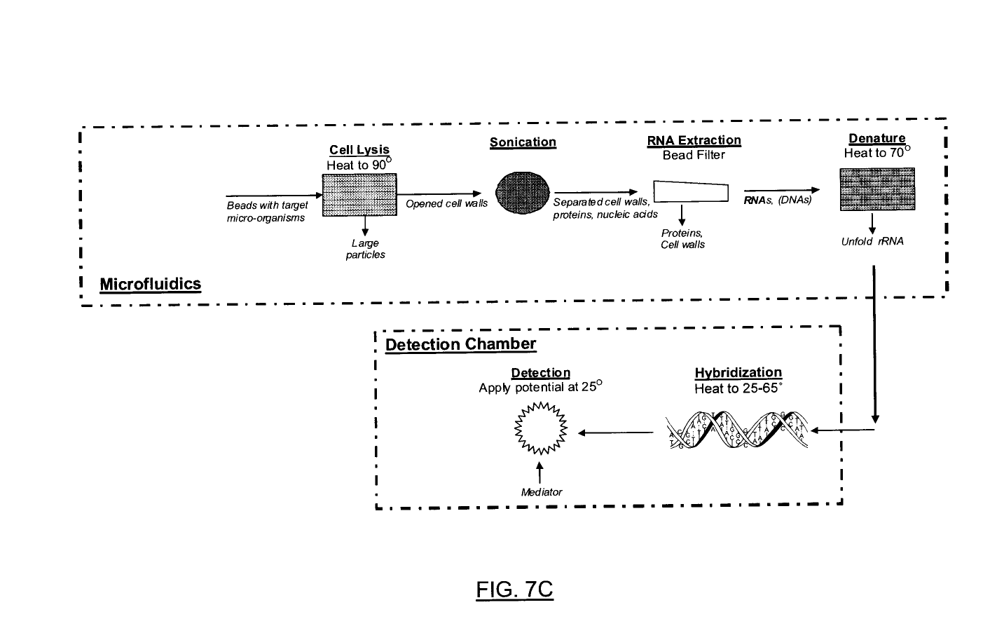

With reference to FIGS. 7A and 7B, there are shown preferred embodiments of a microfluidics module 307 according to a preferred embodiment of the invention.

The microfluidics module generally includes a first microfluidics assembly 312, a filter housing 316 and a second microfluidics assembly 309.

Referring to FIG. 7A, in the illustrated embodiment the incoming magnetic bead condensate is fed to an input chamber 402 and then to different components of the first microfluidics assembly, where processes and additives can be applied. In some applications, the incoming magnetic bead condensate is separated into two portions with the first portion processed immediately and delivered to an unused biochip to measure total cells; and the second portion treated with a growth medium, heat and other processes for several minutes or hours to allow viable cells to reproduce and then be processed. In this case, the incoming magnetic bead condensate is fed from the magnetic bead separation module 306 to a metering system 401 and is divided evenly into input chamber 402a and input chamber 402b. The first microfluidics assembly 312 preferably includes first and second branches 403a and 403b, each including the same processing components as will be explained in detail further below. The second branch 403b additionally includes growth means for causing viable cells in the target biomolecules of the second portion of the beaded biomolecule condensate to reproduce prior to being processed. Preferably, the growth means include a culture chamber 408 which receives the second portion of the beaded biomolecule condensate from the metering system 401, through the second input chamber 402b, and nutrient providing means for providing nutrients to the culture chamber 408. The nutrients or growth medium may for example be a lactose broth. These nutrients may be stored in a dispenser 418a in fluid communication with the culture chamber 408. A heater 410 for heating the culture chamber may apply heat according to appropriate growth conditions for the biomolecules under study, for example at 35-37° C. Agitation may additionally take place. The second portion of the beaded biomolecule condensate may remain in the culture chamber 408 for a predetermined period of time, such as 1 to 2 hours depending on the characteristics of the target biomolecules. In an alternative embodiment, the first microfluidics assembly may include a single branch which may be sanitized between the processing of the first and second portions of the beaded biomolecule condensate.

In both branches of the first microfluidics assembly 312, or in a single branch, as the case may be, cell lysing means are provided for lysing cells attached to the magnetic beads of the beaded biomolecule condensate. The lysing releases the biomolecule constituents of the target biomolecules. Preferably, a lysis mixing chamber 404a, 404b is provided, in which the beaded biomolecule condensate is mixed with cell lysis reagents, such as a Bacterial Protect Reagent (BPR) solution or a lysozyme lysing solution. Appropriate solutions may for example be obtained from the company Qiagen suh as a RLT (trademark) buffer solution. Each solution reagent may be provided from a suitable dispenser 418b in fluid communication with the corresponding mixing chamber 404a, 404b. The solution containing the mixed beaded biomolecule condensate and cell lysis reagents is agitated back and forth in the lysis mixing chamber and then sent to extraction chamber 406a. A heater 410 preferably collaborates with the lysis mixing chamber 404a, 404b, to heat this chamber to an optimum temperature in the range of 25 to 37° C., preferably for 5 to 10 minutes, should it be necessary to heat the sample to facilitate cell lysis. Of course, other temperature ranges or heating times may be considered depending on the particular application. For example, temperatures as high as 90° C. can be reached depending on the lysis method used. This process opens the cell walls of the target biomolecules, exposing the RNA strands therein. Optionally, the beaded biomolecule is further processed to help separate the biomolecule constituents from their cells. In one embodiment, a sonication device (not shown) projects ultrasonic energy through the mixing chamber. Other methods can also be employed to open the cell walls and extract the target biomolecule constituents used for biodetection. For example, the beaded biomolecule condensate can be submitted to an alternating low and high temperature cycle to break open cell walls. This technique may for example be employed for cryptosporidium oocysts that may be more resistant to conventional cell lysis. This process may take place in an additional mixing chamber and the same heater as mentioned above or a different one may be provided for this purpose.

Still referring to the embodiment of FIG. 7A, once the cell lysis is completed, the resulting beaded biomolecule condensate, in which the biomolecule constituents are now separate from the magnetic beads and other waste material from the cell lysis, must be filtered, preferably through a filter membrane 310, which could for example be made of silica. As the biomolecule constituents are very small, they are allowed though the filter 310, whereas the waste materials from the cell lysis are retained. As such filter membranes 310 are usually one-time use components, the microfluidics module 307 preferably includes several of them, provided in a filter housing 316. A first distribution manifold 311a sends the sample to an unused filter membrane 310. The first distribution manifold 311a is in fluid communication with the first microfluidics assembly 312 to receive the beaded biomolecule condensate therefrom, and a plurality of outlets, each connected to a corresponding one of the filter membrane's first control means, enable the controlled directing of the concentrated biomolecule condensate to any one of these outlets. In one embodiment, the manifold 311a can be constructed as several layers of capillaries and potentially compressed air or other gas controlled membrane switches where the capillaries on one layer are provided with a default and optional direction for the sample to flow to the underlying layer. The direction could be set by a controller 414 using pneumatics, or an electrical or other switching mechanism.

The filtered biomolecule constituents are then processed through a second microfluidics assembly, which includes preparation means for the preparation of the filtered biomolecule constituents for detection. For this purpose, one or more mixing chamber 412 is provided. Each mixing chamber 412 mixes the biomolecule constituents with an appropriate additive, such as an elution buffer, binding buffer or washing buffer, Appropriate solutions may for example be as provided from the company Qiagen such as a RW buffer, a DNase buffer, a DNase and RDD solution, a RPE buffer (all trademarks?) and an Ethanol solution. Preferably, the mixture in each mixing chamber 412 is agitated back and forth, preferably through alternative pumping means (not shown). Heating means such as an additional heater 411 may be provided for heating the biomolecule constituents at an appropriate temperature and for a length of time sufficient to denature and unfold the RNA strands therein. In one embodiment, the biomolecule constituents are heated to about 70° C. to permit denaturing and unfolding of target constituents such as rRNA.

The biomolecule constituents are then delivered to an unused one-time use biosensing device 200 for measuring the total number of target cells. A second distribution manifold 311b, in fluid communication with the second microfluidics assembly 309, receives the biomolecule constituents therefrom, and distributes them to one of a plurality of outlets, each connected to a corresponding biosensing device 200. Any waste output is sent to a waste container. In another embodiment, the RNA output is passed to an unused biosensing device and is agitated back and forth and heated to 25-65° C. for hybridization. A mediator and positive control target are added to the solution and a detection process commences at about 25° C.

Although the first microfluidics assembly 312, first manifold 311a, filter housing 316, second microfluidics assembly 309, second manifold 311b and biosensing devices 200 are shown in FIG. 7A as consecutive layers, one skilled in the art will understand that in practice, these components may be distributed differently. In one embodiment, the microfluidics module 307 preferably includes a reusable assembly and a one-time use assembly. Advantageously, the reusable assembly can be sanitized after each use. Preferably, the one-time use assembly includes components which cannot be sanitized for further use. The one-time use components may for example include the biosensing devices themselves, and the filter membranes. Preferably, the first microfluidics assembly, second microfluidics assembly and manifold or manifolds are all part of the reusable assembly. This division can simplify the volume production of one-time use microfluidics components, which are typically produced in higher number than the reusable microfluidics, thus providing further cost efficiencies in the volume production.

Referring to FIG. 7B, there is shown another embodiment of the invention where the components of the second microfluidics assembly are packaged with the biosensing devices as duplexes and are also referred to as the “biochip”. Various fluids can be added to the microfluidics module or biochips, such as lysis reagents, elution buffers, binding buffers, and washing buffers. All fluids, and the growth medium, can be inserted in reservoirs on the reusable or one-time use microfluidics assemblies, or stored in separate dispensers 418a, 418b, etc. outside the microfluidics, and then added through input wells or the manifold. In the latter case, the dispensers can be refilled when replacing the one-time use assembly. A replacement cartridge is needed after all of the biochips on the cartridge are used.

In one embodiment, a chemical mediator for amplifying the electrochemical signal from the biosensing device and target biomolecules for the positive control electrodes, is also available for adding to the biosensing device. The different steps of this process are shown in FIG. 7C.

Alternatively, the components of the second microfluidics assembly may be integrated at the level of the filter membranes or the manifold.

A delivery system for moving liquid though the microfluidics module can include a pump 416 to provide compressed gas to push fluids through the module, and a vacuum 419 that can pull fluids through the microfluidics module. The pump 416 can also be used to control pneumatic switches 414 in the manifold.

In another embodiment, a cartridge houses a plurality of biochips, or a plurality of one-time use microfluidics assemblies and one-time use biosensing devices, also referred to herein as an insert. For example, the cartridge could have a vertical stack of inserts similar to a PEZ (trademark) candy dispenser, with an unused insert placed from one end into a housing to receive the magnetic bead condensate and then replaced after use. In another embodiment, inserts are loaded into a circular carousel resembling a 35 mm slide projector, where an unused insert is placed into a housing from the carousel to receive the microfluidics condensate and then replaced after use.

In the illustrated embodiment, in accordance with one aspect of the invention, a set of one-time-use components for the microfluidics module of a condensing device as described herein may be provided. This set may include a filter component holding the filter housing which includes a plurality of filter membranes, each having a corresponding outlet. A separate sensor component may host a plurality of said biosensing devices in equal number to the filter membranes. The two components may, for example, take the form of a disk and be provided individually, or held in a fixed arrangement through appropriate holding means. In one embodiment, each biosensor and each membrane is provided on a biochip manually inserted into a structure to receive the processed biomolecule constituents. The filter component and sensor component are preferably fabricated on separate undiced substrates, such as wafers. This embodiment has the advantage of minimal moving parts that may cause electrical interference or additional maintenance. Furthermore, the ability to fabricate and deliver multiple inserts or biochips of microfluidics and biosensing device duplexes on undiced wafers is enabled by a novel fabrication technique for the volume production of biochips that provides cost efficiencies in the volume production of semiconductors, shared circuitry to reduce the number of connectors and simplified packaging, which as one skilled in the art will recognize, provides significant cost reductions over the fabrication and packaging of individual biochips. This will be further described below.

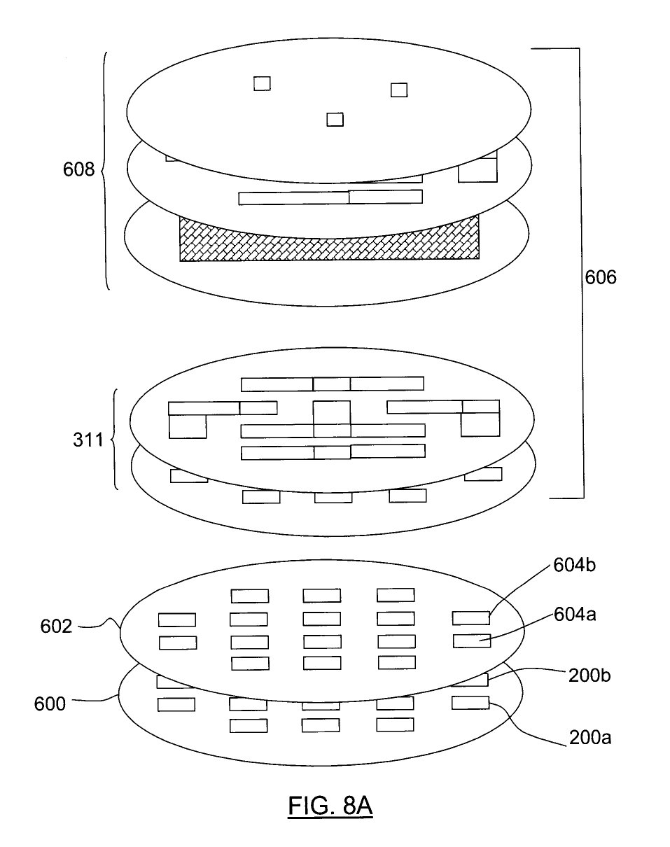

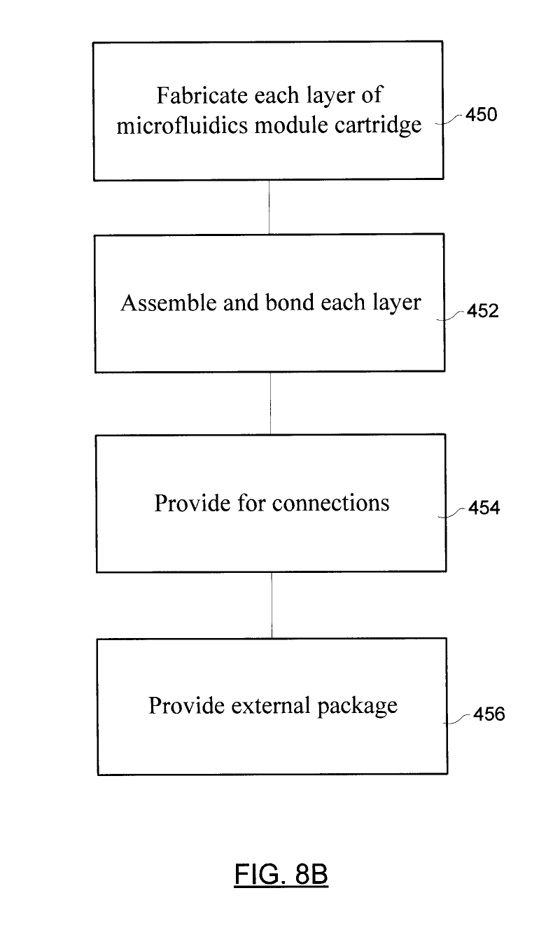

Referring to FIG. 8A, the microfluidics module may be made of three groups of layers: the bottom layer or layers 600, including one or more one-time use biosensing devices 200a, 200b etc; the middle layer or layers 602, comprising one or more one-time use microfluidics assemblies 604a, 604b, etc each of which are aligned with corresponding biosensing devices 200a, 200b etc to form biochip duplexes; and the top layer or layers 606, comprising reusable microfluidics components 608. The top layer 606 can contain a manifold 311 to distribute RNA and/or other biomolecule constituents to the biochip duplexes, a heater and other required components to support the necessary processes.

All or some of the layers of FIG. 8A preferably form a cartridge that can be replaced when all of the one-time use microfluidics assemblies and biosensing devices have been used. The replaceable cartridge can include the entire microfluidics module 307 or just the bottom and middle layers 600 and 602 respectively hosting the one-time use microfluidics 604 and biosensing devices 200.

With reference to FIG. 8B, there is shown a flow chart generally illustrating a method of fabricating a microfluidics module such as the one shown in FIG. 8A. The method first includes fabricating 450 the bottom and middle layers. In the embodiment using biosensing devices as shown in FIG. 2, the bottom layer with the biosensing devices is typically the most sophisticated and therefore the most difficult to fabricate, as it can require the use of a multitude of identical dies of one-time use biosensing devices with nanometer scale features. For example the Assignee's U.S. patent application Ser. No. 12/216,914 can employ millions of nanoscale electrodes and biomolecular probes that are patterned using nanopatterning techniques such as NanoImprint Lithography—Hot Embossing, or photolithography and related fabricating processes. The resulting bottom layer defines a substrate such as a silicon wafer containing a plurality of one-time use biosensing devices at predetermined coordinates on the substrate, as is typically done in volume production used in semiconductor fabrication. However, the last and typically most expensive processes for converting the wafer into individual chips through dicing, adding connectors and packaging are intentionally not employed on the bottom layer, in order to keep all of the one-time use biosensing devices on the same wafer without dicing, connectors or packaging.

In the preferred embodiment, the middle layer defines one or more substrates containing a plurality of one-time use microfluidics assemblies at predetermined coordinates on the substrate. The middle layer makes use of a multitude of dies of one-time use microfluidics with microscale features and can be fabricated on multiple types of water-proof materials, preferably a thermal polymer or machinable polymer, which is low cost, easy to align and bond with the bottom layer, and readily mass produced, preferably using NanoImprint Lithography or photolithography, depending on the critical dimensions of the nano-electrodes, or micromachining or injection molding, and/or other processes.

The top layer defines one or more substrates containing a single reusable microfluidics assembly and distribution manifold with microscale features, and can be fabricated on multiple types of water-proof materials, preferably a thermal polymer or machinable polymer, which is low cost, easy to align and bond with the bottom layer, and readily mass produced using Lithography, micromachining, injection molding, and/or other processes.

The method then includes aligning the bottom and middle layers and then attaching 452 them to each other so that the one-time use biosensing devices and microfluidics assemblies are provided as duplexed biochips. The top layer, containing the reusable portion of the microfluidics assembly, is then also aligned and attached.

The method preferably next includes providing for connections 454 associated with electrical connections for each biosensing device, fluid input and output, compressed gas and control mechanism for the manifold, as well as packaging 456 the resulting cartridge in view of its intended use.

### Sanitizing Method

In accordance with another aspect of the invention, a sanitizing method is provided that allows for a fully automated or semi automated process to sanitize the reusable modules of a condensation device according to at least some of the embodiments above, between uses.

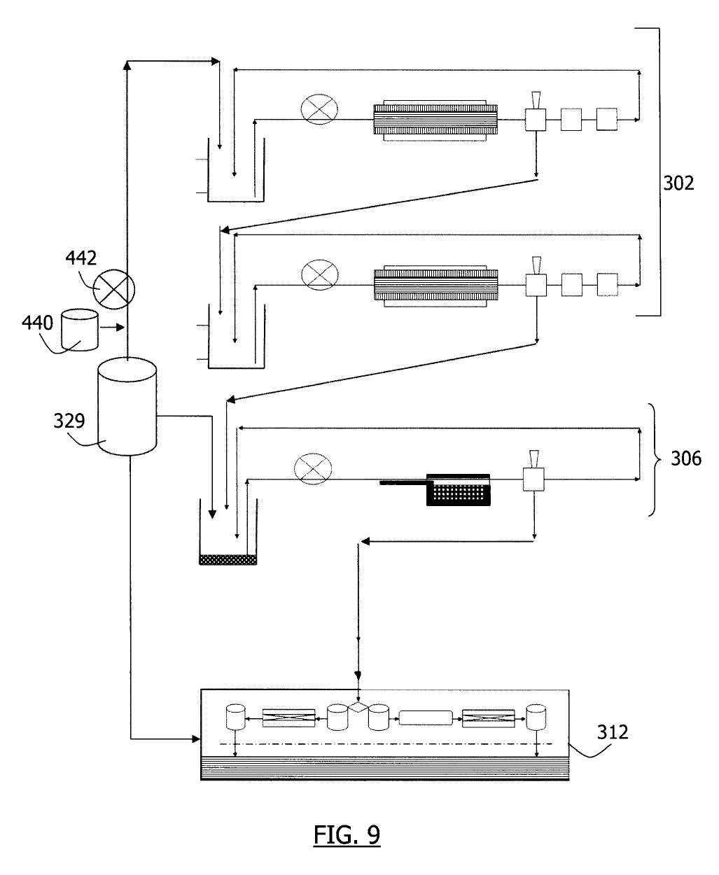

Referring to FIG. 9, in a preferred embodiment, the sanitization method makes use of the filtered liquid reservoir 329 used for collecting filtered liquid that has been processed by the filtration modules to be free of viruses, bacteria and protozoa; a dispenser 440 connected to the filtered liquid reservoir 326 that can add a sanitizing agent to the filtered liquid to create a sanitizing solution; and a pumping system 442 that can circulate the sanitizing solution through any, either or all reusable modules and systems of the condensing device including pipes, tanks, filters and components in the filtration module, magnetic beads separation module and reusable portion of the microfluidics module that may have had contact with the liquid solution or condensate containing target biomolecules. The sanitizing solution is left therein for a soaking period.

In one embodiment, the sanitizing solution comprises filtered water that has been filtered by the primary and secondary filtration systems, and the sanitizing agent is 30 mL of 35% hydrogen peroxide per 10 L of filtered water. However, other filtered liquids, sanitizing agents and concentrations can be used depending on the input media, target biomolecules and other materials in the liquid solution. The sanitizing method's pumps, pipes, valves and other components can be configured to support the sanitizing method automatically with Programmable Logic Controllers (PLCs) or manually by an operator to direct and control the flow of the sanitizing liquid and sanitizing agent. A dispenser releases the sanitizing agent into the filtered solution as the solution is being pumped from its reservoir.

Once the condensing device is filled with the sanitizing solution, a predetermined contact time is preferably measured to permit the sanitizing solution to adequately disinfect the reusable components before the next biodetection test. In the above embodiment, the minimum contact time is 60 minutes. After the minimum contact time is reached, the pumping system 442 is used to pump the sanitizing solution out of the modules and empty the systems. The discharge can be sent to a drainage discharge or to a carboy.

The system is then prepared for the next detection test, as per appropriate maintenance procedures. In one embodiment, the sanitizing solution can be left in the ultrafiltration filters for a storage period, as such filters can degrade when in contact with air. Alternatively, the filters can be filled with a different liquid soaking solution and flowmeters can be reset to zero.

### Other Configurations and Applications

It should be noted that the condensing device according to embodiments of the present invention may advantageously, although not limitatively, be used with the abovementioned biosensing device in a biohazard early warning system for the fully-automated or semi-automated sampling, condensing, and detection of pathogenic bacteria, viruses and protozoa, and other target biomolecules in water, food, air, surfaces, insects, animals, and humans in a sensor network or portable device. Without the need for time-intensive sampling, incubation and amplification techniques, less time is needed to identify a potential pathogen outbreak and provide the appropriate response to stop the transmission.

However, many other configurations and related applications can also be devised without departing from the scope of the invention. For example, the condensation device can replace PCR or other incubation and amplification techniques in screening specific genes for unknown mutations and in genotyping using known sequence-tagged site (STS) markers for medical testing, drug discovery and other biological applications. As well, the condensing device can be used for chemical sensors and other sensing applications in addition to biosensing devices

The condensing device can be used with all of its modules as described in the embodiments and figures, or alternatively can be effective in certain applications when some of the modules are streamlined or removed. For example, when detecting target biomolecules known to be in very high concentrations in a solution, such as in sewage waste water or recreational beach water, then a much smaller sample may be sufficient for detection accuracy. In these cases the filtration module and/or the magnetic bead separation module may not be needed, and can be omitted from the configuration to reduce the processing time. Furthermore, it is anticipated that the various new sensing technologies and enhancements will improve sensing sensitivity and specificity, making condensing less required.

In other cases, the biochip may be used in a rapid screening mode, which tolerates a much greater range of false positive and false negative results than the diagnostic mode described in the embodiments above, in order to have preliminary test results in minutes rather than hours. When the biochips are used in a screening mode in a handheld device or wearable sensor or in front of the diagnostic device with a biomolecule condensing device, then the condensing device modules may be further streamlined or omitted.

Finally, other types of modules can be employed in combination with the condensing device to collect and liquefy solids in air, or from insects, food, tissues, feces, and other solid materials.

Of course, numerous other modifications could be made to the embodiments above without departing from the scope of the present invention.

1. A biomolecule condensing device for providing a concentrated biomolecule condensate to at least one biosensing device, the concentrated biomolecule condensate being obtained from a fluid sample potentially containing traces of at least one target biomolecule, the biomolecule condensing device comprising:
a filtration module comprising at least one ultrafiltration assembly for separating said fluid sample into a filtered liquid and a retentate biomolecule condensate containing at least one of said target biomolecule if present in the fluid sample;
a magnetic bead separation module for separating the retentate biomolecule condensate into a beaded biomolecule condensate containing said target biomolecules and waste materials, said magnetic bead separation module comprising magnetic beads coated with antibodies of the at least one target biomolecule so that the target biomolecules in the retentate biomolecule condensate become attached to said magnetic beads; and
a microfluidics module for processing the beaded biomolecule condensate to extract constituents of said target biomolecules therefrom, thereby obtaining the concentrated biomolecule condensate, said microfluidics module enabling the distribution of said concentrated biomolecule condensate to one of the at least one biosensing device.

2. The biomolecule condensing device according to claim 1, wherein each ultrafiltration assembly of the filtration module comprises:
a) a sample reservoir;
b) a filter housing containing an ultrafiltration filter for separating the filtered liquid and retentate biomolecule condensate, the filter housing having an inlet in fluid communication with said sample reservoir, a liquid outlet for outputting the filtered liquid, and a retentate outlet for outputting the retentate biomolecule condensate;
c) a concentration loop for circulating the retentate condensate from the retentate outlet of the filter housing back to the sample reservoir and further circulating the retentate condensate through the filter housing for multiple passes, additional portions of said filtered liquid being removed therefrom at each pass; and
d) an extraction line for extracting the retentate biomolecule condensate out of said ultrafiltration assembly after said multiple passes.

3. The biomolecule condensing device according to claim 2, wherein the ultrafiltration filter of each ultrafiltration assembly of the filtration module is a hollow fiber tangential flow filter.

4. The biomolecule condensing device according to claim 2, wherein the concentration loop of each ultrafiltration assembly comprises:
an inlet line connecting the sample reservoir and the inlet of the filter housing;
an outlet line connecting the condensate outlet of the filter housing to the sample reservoir; and
a pump for cyclically circulating the fluid sample through said concentration loop.

5. The biomolecule condensing device according to claim 2, wherein each ultrafiltration assembly comprises a 3-way valve having an inlet in fluid communication with the retentate outlet of the filter housing, a first outlet in fluid communication with the sample reservoir, and a second outlet connected to said extraction line.

6. The biomolecule condensing device according to claim 5, wherein each ultrafiltration assembly comprises a sensor in the sample reservoir for sensing a fluid level therein, the sensor being operationally connected to the 3-way valve to activate the second outlet thereof when said fluid level drops below a lower threshold level.

7. The biomolecule condensing device according to claim 2, wherein the filtration module further comprises a filtered liquid reservoir connected to the filtered liquid outlet of the filter housing of the at least one ultrafiltration assembly to receive the filtered liquid therefrom.

8. The biomolecule condensing device according to claim 2, wherein the filtration module comprises a primary and secondary said ultrafiltration assembly, said primary and secondary ultrafiltration assemblies being connected in a series to provide the retentate biomolecule concentrate extracted from the primary ultrafiltration assembly to the sample reservoir of the secondary ultrafiltration assembly.

9. The biomolecule condensing device according to claim 1, wherein the filtration module comprises a clump-breaking mechanism for breaking up aggregate clumps or biofilms in the fluid sample.

10. The biomolecule condensing device according to claim 9, wherein the clump-breaking mechanism comprises a hydrodynamic cavitation device or a sonication device.

11. The biomolecule condensing device according to claim 1, wherein the filtration module further comprises at least one chemical dispensing device for dispensing chemicals in the fluid sample.

12. The biomolecule condensing device according to claim 11, wherein the chemicals comprise at least one of sodium polysulfide and sodium thiosulfide.

13. The biomolecule condensing device according to claim 1, wherein the filtration module further comprises at least one pre-processing filter for removing unwanted materials from the fluid sample.

14. The biomolecule condensing device according to claim 13, wherein said at least one pre-processing filter comprises at least one of a large mesh filter and a carbon filter.

15. The biomolecule condensing device according to claim 1, wherein the filtration module comprises a pre-filtration module upstream of said at least one ultrafiltration assembly for processing said fluid sample, said pre-filtration module comprising at least one of a clump-breaking mechanism, a chemical dispensing device and a pre-processing filter.

16. The biomolecule condensing device according to claim 1, wherein the magnetic bead separation module comprises:
a condensation chamber for receiving the retentate biomolecule condensate and the magnetic beads therein, thereby promoting the attachment of the target biomolecules in the retentate biomolecule condensate to the ones of the magnetic beads coated with the corresponding antibodies; and
magnetization means for magnetically retaining the magnetic beads within said condensation chamber while removing a remainder of the retentate biomolecule condensate therefrom.

17. The biomolecule condensing device according to claim 16, wherein the condensation chamber of the magnetic beads separation module receives a plurality of types of said magnetic beads, each type being coated with antibodies of a different one of said target biomolecules.

18. The biomolecule condensing device according to claim 16, wherein the magnetic bead separation module further comprises a mixing tank for mixing the retentate biomolecule condensate and magnetic beads together into a magnetic bead mixture, the mixing tank being connected to the condensation chamber to provide said magnetic bead mixture thereto.

19. The biomolecule condensing device according to claim 18, wherein the magnetic beads separation module further comprises at least one solution reservoir, each containing a solution, each of said at least one reservoir being in fluid communication with the mixing tank for providing the corresponding solution therein, each of said at least one solution being included to the magnetic bead mixture.

20. The biomolecule condensing device according to claim 19, wherein the solution contained in each of said at least one solution reservoir is selected from the group comprising the filtered liquid separated from the fluid sample by the filtration module, a buffer solution, a re-suspension solution and a combination thereof.

21. The biomolecule condensing device according to claim 18, wherein the magnetic bead separation module further comprises a recirculation assembly for circulating the magnetic bead mixture through the condensation chamber for a plurality of passes.

22. The biomolecule condensing device according to claim 16, wherein the magnetization means comprises a permanent magnet mounted adjacent to the condensation chamber

23. The biomolecule condensing device according to claim 16, wherein the magnetization means comprises a coil assembly mounted around said condensation chamber for inducing a magnetic field within said condensation chamber.

24. The biomolecule condensing device according to claim 16, wherein the condensation chamber comprises a waste outlet for extracting therefrom a waste solution resulting from said magnetic bead separation, the magnetic bead separation module further comprising a waste reservoir in fluid communication with said waste outlet for receiving the waste solution therefrom.

25. The biomolecule condensing device according to claim 16, further comprising an extraction line connected to the condensation chamber for pumping the beaded biomolecule concentrate therefrom towards the microfluidics module.

26. The biomolecule condensing device according to claim 1, wherein the microfluidics module comprises:
a first microfluidics assembly comprising cell lysing means for lysing cells attached to the magnetic beads of the beaded biomolecule condensate, said lysing releasing said biomolecule constituents of said target biomolecules;
a filter housing receiving the beaded biomolecule condensate from the first microfluidics assembly, said filter housing containing at least one filter membrane for retaining waste material from said cell lysis and allowing said biomolecule constituents therethrough; and
a second microfluidics assembly receiving the biomolecule constituents from the filter housing and comprising preparation means for the preparation of said biomolecule constituents for detection.

27. The biomolecule condensing device according to claim 26, wherein the cell lysing means comprise a lysis mixing chamber for mixing the beaded biomolecule condensate with cell lysis reagents.

28. The biomolecule condensing device according to claim 27, wherein said cell lysis reagents comprise at least one of a bacterial protect reagent solution, a lysozyme lysing solution and a buffer solution.

29. The biomolecule condensing device according to claim 27, wherein the cell lysis means comprise a heater collaborating with the lysis mixing chamber, said heater being controllable for heating said lysis mixing chamber to an optimum temperature in the range of 25 to 37° C. for 5 to 10 minutes.

30. The biomolecule condensing device according to claim 27, wherein the first microfluidics assembly comprises a sonication device for projecting ultrasonic energy through said mixing chamber.

31. The biomolecule condensing device according to claim 27, wherein the first microfluidics assembly comprises means for submitting the beaded biomolecule condensate to an alternating low and high temperature cycle.

32. The biomolecule condensing device according to claim 26, wherein the preparation means of the second microfluidics assembly comprises at least one mixing chamber, each mixing chamber mixing said biomolecule constituents with at least one of a RW buffer, a DNase buffer, a DNase and RDD solution, a RPE buffer and an Ethanol solution.

33. The biomolecule condensing device according to claim 26, wherein the preparation means of the second microfluidics assembly comprises heating means for heating said biomolecule constituents at a temperature and time sufficient to denature RNA strands therein.

34. The biomolecule condensing device according to claim 32, wherein at least one of the mixing chambers of the second microfluidics assembly receives and mixes the biomolecule constituents with at least one of a wash buffer and a mediator, said at least one of the mixing chambers of the second microfluidics assembly being in fluid communication with said biosensing device to deliver said biomolecule constituents thereto.

35. The biomolecule condensing device according to claim 26, wherein the microfluidics module comprises a metering system for receiving the magnetic bead concentrate from the magnetic bead separation module and for dividing the beaded biomolecule condensate into first and second portions thereof; and
wherein the first microfluidics assembly has first and second branches for separately processing said first and second portions of the beaded biomolecule condensate, the second branch of the microfluidics assembly comprising growth means for causing viable cells in said target biomolecules of the second portion of the beaded biomolecule condensate to reproduce prior to said processing.

36. The biomolecule condensing device according to claim 35, wherein said growth means comprise:
a culture chamber receiving said second portion of the beaded biomolecule condensate from the metering system
a nutrient providing means for providing nutrients to said culture chamber; and
a mixing means for mixing the contents of said culture chamber.

37. The biomolecule condensing device according to claim 36, wherein said growth means further comprise heating means for heating said culture chamber.

38. The biomolecule condensing device according to claim 1, wherein the microfluidics module comprises a reusable assembly comprising a portion of said microfluidics components, and a one-time use assembly comprising a remaining portion of said microfluidics components and said at least one biosensing device.

39. The biomolecule condensing device according to claim 38, wherein said reusable assembly comprises:
a metering system for dividing the beaded biomolecule condensate in said input chamber into first and second portions thereof, the metering system having a first and a second output; and
a first microfluidics assembly in fluid communication with the metering system for receiving the first biomolecule condensate portion therefrom, the first microfluidics assembly comprising first and second branches each comprising cell lysing means for lysing cells attached to the magnetic beads of the beaded biomolecule condensate, said lysing releasing said biomolecule constituents of said target biomolecules, said second branch further comprising growth means for causing viable cells in said target biomolecules of the second portion of the beaded biomolecule condensate to reproduce prior to said processing, said growth means comprising a culture chamber receiving said second portion of the beaded biomolecule condensate from the metering system, nutrient providing means for providing nutrients to said culture chamber and heating means for heating said culture chamber.

40. The biomolecule condensing device according to claim 39, wherein said one-time use assembly comprises a filter housing containing at least one filter membrane for retaining waste material from said cell lysis and allowing said biomolecule constituents therethrough.

41. The biomolecule condensing device according to claim 40, wherein the filter housing comprises a plurality of said filter membranes, and the reusable assembly comprises:
a first distribution manifold in fluid communication with the first microfluidics assembly to receive the beaded biomolecule condensate therefrom, and a plurality of outlets, each connected to a corresponding one of said filter membranes; and
first control means enabling the controlled directing of said concentrated biomolecule condensate to any one of said outlets of the first distribution manifold.

42. The biomolecule condensing device according to claim 41, wherein the reusable assembly comprises a second microfluidics assembly receiving the biomolecule constituents from the filter housing and comprising preparation means for the preparation of said biomolecule constituents for detection.

43. The biomolecule condensing device according to claim 42, wherein the reusable microfluidics assembly comprises:
a second distribution manifold in fluid communication with the second microfluidics assembly to receive the biomolecule constituents therefrom, and a plurality of outlets, each connected to a corresponding one of said biosensing devices; and
second control means enabling the controlled directing of said biomolecule constituents to any one of said outlets of the second distribution manifold.

44. A set of one-time-use components for the microfluidics module of the condensing device according to claim 26, comprising:
a filter component comprising said filter housing, said filter housing comprising a plurality of said filter membranes, each having a corresponding outlet; and
a sensor component comprising a plurality of said biosensing devices in equal number to said plurality of filter membranes.

45. The set of one-time use components according to claim 44, comprising holding means holding said filter and sensor components in a fixed arrangement.

46. A method for sanitizing the biomolecule condensing device according to claim 1, comprising:
a) adding a sanitizing agent to the filtered liquid obtained through the filtering of said filtering module, thereby obtaining a sanitizing solution;
b) circulating said sanitizing solution through at least one of the filtration module, the magnetic bead separation module and the microfluidics module; and
c) leaving the sanitizing solution in said at least one of the filtration module, the magnetic bead separation module and the microfluidics module for a soaking period.

47. The method for sanitizing according to claim 46, wherein the sanitizing agent comprises hydrogen peroxide.

48. The method according to claim 46, wherein the circulating of b) comprises circulating said sanitizing solution through at least one ultrafiltration filter in the filtration module, and the soaking period of c) comprises a storage period of said ultrafiltration filters.

49. The method for sanitizing of claim 46, further comprising:
d) removing the sanitizing solution from said at least one of the filtration module, the magnetic bead separation module and the microfluidics module.

50. A condensing method for providing a concentrated biomolecule condensate to at least one biosensing device, the concentrated biomolecule condensate being obtained from a fluid sample potentially containing traces of at least one target biomolecule, the method comprising:
a) separating said fluid sample into a filtered liquid and a retentate biomolecule condensate containing at least one of said target biomolecule if present in the fluid sample;
b) attaching the target biomolecules in the retentate biomolecule condensate to magnetic beads coated with antibodies of the at least one target biomolecule, thereby obtaining a beaded biomolecule condensate, and separating the same from waste materials; and
c) processing the biomolecule condensate to extract constituents of said target biomolecules therefrom, thereby obtaining the concentrated biomolecule condensate, and distributing the same to one of the at least one biosensing device.

51. The condensing method according to claim 50, wherein the separating of a) comprises at least one ultrafiltration cycle, each ultrafiltration cycle comprising:
i. receiving said fluid sample in a sample reservoir;
ii. circulating the fluid sample through an ultrafiltration filter for separating the filtered liquid and retentate biomolecule condensate; and
iii. extracting the retentate biomolecule condensate out of said ultrafiltration assembly.

52. The condensing method according to claim 51, comprising, prior to the extracting of a) ii, circulating the retentate condensate back to the sample reservoir and further circulating the retentate condensate through the ultrafiltration filter for multiple passes, additional portions of said filtered liquid being removed therefrom at each of said multiple passes.

53. The condensing method according to claim 52, further comprising sensing a fluid level in the sample reservoir, and proceeding with the extracting of a)iii when said fluid level drops below a lower threshold level.

54. The condensing method according to claim 50, wherein the separating of a) comprises breaking up aggregate clumps in the fluid sample.

55. The condensing method according to claim 54, wherein the breaking up aggregate clumps comprises using hydrodynamic cavitation or sonication.

56. The condensing method according to claim 54, wherein the breaking up aggregate clumps comprises adding a dispersant chemical to the fluid sample.

57. The condensing method according to claim 51, wherein the separating of a) comprises performing a primary and a secondary of said ultrafiltration cycles, the retentate biomolecule concentrate extracted during the primary ultrafiltration cycle being provided as input to the secondary ultrafiltration cycle.

58. The condensing method according to claim 51, wherein each filtration cycle further comprises storing the filtered liquid into a filtered liquid reservoir.

59. The condensing method according to claim 50, wherein the attaching of b) comprises:
mixing the retentate biomolecule condensate and the magnetic beads together, thereby promoting the attachment of the target biomolecules in the retentate biomolecule condensate to the ones of the magnetic beads coated with the corresponding antibodies; and
magnetically retaining the magnetic beads within a condensation chamber while removing a remainder of the retentate biomolecule condensate therefrom.

60. The condensing method according to claim 59, comprising a plurality of types of said magnetic beads, each type being coated with antibodies of a different one of said target biomolecules.

61. The condensing method according to claim 59, wherein said mixing further comprises adding at least one solution to the retentate biomolecule condensate and the magnetic beads, said at least one solution being selected from the group comprising the filtered liquid separated from the fluid sample by the filtration module, a buffer solution, a re-suspension solution and a combination thereof.

62. The condensing method according to claim 50, wherein the processing of c) comprises lysing cells attached to the magnetic beads of the beaded biomolecule condensate to release said biomolecule constituents thereof.

63. The condensing method according to claim 62, wherein said lysing comprises mixing the beaded biomolecule condensate with cell lysis reagents.

64. The condensing method according to claim 63, wherein said cell lysis reagents comprise at least one of a bacterial protect reagent, a lysozyme lysing solution and a buffer solution.

65. The condensing method according to claim 63, wherein said lysing further comprises heating the mixed beaded biomolecule condensate and cell lysis reagents to an optimum temperature in the range of 25 to 37° C. for 5 to 10 minutes.

66. The condensing method according to claim 65, wherein said lysing further comprises projecting ultrasonic energy through said beaded biomolecule condensate.

67. The condensing method according to claim 65, wherein said lysing further comprises submitting the beaded biomolecule condensate to alternating low and high temperature cycles.

68. The condensing method according to claim 62, wherein processing of c) further comprises filtering said beaded biomolecule condensate subsequently to said cell lysing for separating the biomolecule constituents from said magnetic beads and waste material.

69. The condensing method according to claim 68, comprising preparing of said biomolecule constituents for detection subsequent to said filtering.

70. The condensing method according to claim 69, wherein said preparing comprises mixing said biomolecule constituents with at least one of a RW buffer, a DNase buffer, a DNase and RDD solution, a RPE buffer and an Ethanol solution.

71. The condensing method according to claim 69, wherein said preparing comprises heating said biomolecule constituents at a temperature and time sufficient to denature RNA strands therein.

72. The condensing method according to claim 69, wherein said preparing comprises mixing the biomolecule constituents with at least one of a wash buffer and a mediator.

73. The condensing method according to claim 50, wherein the processing of c) comprises dividing the beaded biomolecule condensate into first and second portions thereof, the first beaded biomolecule condensate portion being processed immediately, and the second beaded biomolecule condensate portion being processed after a predetermined delay.

74. The condensing method according to claim 73, comprising holding said second beaded biomolecule condensate portion in a culture chamber during the predetermine delay.

75. The condensing method according to claim 74, further comprising providing nutrients to said culture chamber and heating said culture chamber during said predetermined delay.

76. A filtration module for providing a retentate analyte condensate from a fluid sample potentially containing traces of at least one analyte, the filtration module comprising at least one ultrafiltration assembly for separating said fluid sample into a filtered liquid and said retentate analyte condensate, each ultrafiltration assembly comprising:
a sample reservoir;
a filter housing containing an ultrafiltration filter for separating the filtered liquid and retentate analyte condensate, the filter housing having an inlet in fluid communication with said sample reservoir, a liquid outlet for outputting the filtered liquid, and a retentate outlet for outputting the retentate analyte condensate;
a concentration loop for circulating the retentate analyte condensate from the retentate outlet of the filter housing back to the sample reservoir and further circulating the retentate analyte condensate through the filter housing for multiple passes, additional portions of said filtered liquid being removed therefrom at each pass; and
an extraction line for extracting the retentate analyte condensate out of said ultrafiltration assembly after said multiple passes.

77. The filtration module according to claim 76, wherein the ultrafiltration filter of each ultrafiltration assembly of the filtration module is a hollow fiber tangential flow filter.

78. The filtration module according to claim 76, wherein the concentration loop of each ultrafiltration assembly comprises:
an inlet line connecting the sample reservoir and the inlet of the filter housing;
an outlet line connecting the condensate outlet of the filter housing to the sample reservoir; and
a pump for cyclically circulating the fluid sample through said concentration loop.

79. The filtration module according to claim 76, wherein each ultrafiltration assembly comprises a 3-way valve having an inlet in fluid communication with the retentate outlet of the filter housing, a first outlet in fluid communication with the sample reservoir, and a second outlet connected to said extraction line.

80. The filtration module according to claim 79, wherein each ultrafiltration assembly comprises a sensor in the sample reservoir for sensing a fluid level therein, the sensor being operationally connected to the 3-way valve to activate the second outlet thereof when said fluid level drops below a lower threshold level.

81. The filtration module according to claim 76, wherein the filtration module further comprises a filtered liquid reservoir connected to the filtered liquid outlet of the filter housing of the at least one ultrafiltration assembly to receive the filtered liquid therefrom.

82. The filtration module according to claim 76, wherein the filtration module comprises a primary and a secondary said ultrafiltration assembly, said primary and secondary ultrafiltration assemblies being connected in a series to provide the retentate biomolecule concentrate extracted from the primary ultrafiltration assembly to the sample reservoir of the secondary ultrafiltration assembly.

83. The filtration module according to claim 76, comprising a clump-breaking mechanism for breaking up aggregate clumps or biofilms in the fluid sample.

84. The filtration module according to claim 83, wherein the clump-breaking mechanism comprises a hydrodynamic cavitation device or a sonication device.

85. The filtration module according to claim 76, further comprising at least one chemical dispensing device for dispensing chemicals in the fluid sample.

86. The filtration module according to claim 85, wherein the chemicals comprise at least one of sodium polysulfide and sodium thiosulfide.

87. The filtration module according to claim 76, further comprising at least one pre-processing filter for filtering condensates from the fluid sample.

88. The filtration module according to claim 77, wherein said at least one pre-processing filter comprises at least one of a large mesh filter and a carbon filter.

89. The filtration module according to claim 76, comprising a pre-filtration module upstream of said at least one ultrafiltration assembly for processing said fluid sample, said pre-filtration module comprising at least one of a clump-breaking mechanism, a chemical dispensing device and a pre-processing filter.
